(19) United States
(12) Patent Application Publication
(10) Pub. No.: US 2019/0216793 A1
(43) Pub. Date: Jul. 18, 2019

(54) COMPOSITIONS AND METHODS FOR TREATING NEGATIVE SYMPTOMS IN NON-SCHIZOPHRENIC PATIENTS

(71) Applicant: Minerva Neurosciences, Inc., Waltham, MA (US)

(72) Inventors: Remy LUTHRINGER, Geneva (CH); Michael DAVIDSON, Tel Aviv (IL)

(21) Appl. No.: 16/302,722

(22) PCT Filed: May 23, 2017
(86) PCT No.: PCT/US2017/034030

§ 371 Date: Nov. 19, 2018

Related U.S. Application Data
(60) Provisional application No. 62/341,590, filed on May 25, 2016.

(51) Int. Cl. A61K 31/454; A61P 25/18

(52) U.S. Cl. CPC A61K 31/454; A61P 25/18

(57) ABSTRACT

The present disclosure describes compositions and methods for treating at least one negative symptom in a human subject who does not have a clinical diagnosis of schizophrenia. The compositions and methods employ a therapeutically effective amount of a compound of formula (I), or a pharmaceutically acceptable salt, hydrate, solvate or polymorph thereof.

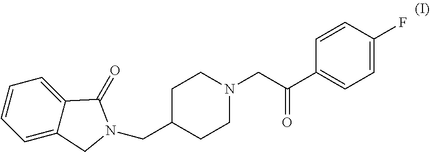 [SMILES: O=C(CN1CCC(CN2Cc3ccccc3C2=O)CC1)c1ccc(F)cc1]

### RELATED APPLICATIONS

This application claims priority to, and the benefit of, U.S. Provisional Application No. 62/341,590, filed May 25, 2016, the entire content of which is incorporated herein by reference in its entirety.

### FIELD OF THE DISCLOSURE

The present disclosure in some embodiments relates generally to compositions and methods for treating negative symptoms, and more specifically to treating negative symptoms in patients who do not have a clinical diagnosis of schizophrenia, i.e., non-schizophrenic patients.

### BACKGROUND

Negative symptoms generally refer to a reduction in normal functioning, and include five major sub-domains: blunted affect (affective flattening, blunted expression), alogia (poverty of speech), amotivation (loss of volition), anhedonia (reduced ability to experience or anticipate pleasure) and asociality (social withdrawal). While negative symptoms are a well-documented and intensively studied aspect of schizophrenia, this class of symptoms has been identified in patients with other psychiatric and neurological disorders, including, for example, Alzheimer's disease and other dementias, particularly frontotemporal dementia (FTD), autism spectrum disorder (ASD), bipolar disorder (BPD), major depressive disorder (MDD), Parkinson's disease, temporal lobe epilepsy, stroke, and traumatic brain injury (TBI) (see, e.g., Boone et al, J. of Internat. Neuropsycol. Soc., 2003, Vol 9, pages 698-709; Bastiaansen, J. et al., J. Autism Dev. Disord. 2011, Vol 41:1256-1266; Getz, K. et al., Am. J. Psychiatry 2002, Vol 159:644-651; Winograd-Gurvich, C. et al., Brain Res. Bulletin, 2006, Vol. 70:312-321; Galynker et al., Neuropsychiatry Neuropsychol Behav Neurol 2000, Vol 13:171-176; Galynker I, et al., J. Nerv. Ment. Dis 1997, Vol 185:616-621; Chaudhury, S., et al., Indian J. of Neurotrauma 2005, Vol 2:13-21; Ameen, S et al., German J. of Psychiatry 2007). Indeed, as early as 2001, it was proposed that negative symptoms are common to mental illnesses generally (Herbener and Harrow, Schizophrenia Bulletin 2001, Vol. 27:527-537). Furthermore, reports of several population studies have concluded that between 20-22% of the general population have one or more negative symptoms, and that the majority of subjects with negative symptoms do not exhibit a clinical diagnosed psychiatric disorder (Werbeloff, N. et al., PLoS ONE 2015, Vol 10:e0119852; Barrantes-Vidal, N., et al., Schizophr. Res. 2010, Vol 122:219-225).

At present, no effective treatments have been approved to treat negative symptoms in schizophrenia or in any other mental disease or neurological condition.

### SUMMARY

The present disclosure is based, in part, on the results of a prospective Phase IIb, 12-week, randomized, double-blind, placebo-controlled parallel clinical trial, which demonstrated a statistically significant benefit of 32 mg and 64 mg doses of MIN-101 over placebo in improving negative symptoms in a cohort of 244 schizophrenic patients with negative symptoms. During this 12-week trial, positive symptoms remained stable and extrapyramidal symptoms (EPS) were absent, consistent with the notion that MIN-101 has a direct and specific effect on negative symptoms rather than improvements on other symptoms. MIN-101 is under clinical development by Minerva Neurosciences (Waltham, Mass.) for the treatment of negative symptoms in schizophrenia.

The active compound in MIN-101 (previously known as CYR-101 and MT-210) has the chemical name 2-{1-[2-(4-Fluorophenyl)-2-oxoethyl]piperidin-4-ylmethyl}-2,3-dihydroisoindol-1-one monohydrochloride dihydrate. The structure of the free base is the compound of formula I (Compound I):

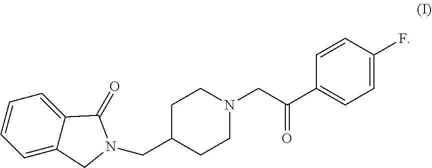 [SMILES: O=C(CN1CCC(CN2Cc3ccccc3C2=O)CC1)c1ccc(F)cc1]

Compound I has specific affinities for sigma₂, 5-hydroxytryptamine-2A (5-HT2A) and at lower affinity levels, α1-adrenergic receptors. MIN-101 exhibits very low or no affinity for other receptors including dopaminergic, muscarinic, cholinergic, and histaminergic receptors. In vivo functional studies have established that MIN-101 is an antagonist at both 5-HT2A and sigma₂ receptors. Two main metabolites of Compound I have been identified and named BFB-520 and BFB-999. The BFB-520 metabolite has been associated with prolongation of QT intervals at supra-therapeutic levels.

In one aspect, the disclosure provides a composition comprising a compound of formula (I) (Compound I), or a pharmaceutically acceptable salt, hydrate, solvate or polymorph thereof, for use in a method of treating at least one negative symptom in a non-schizophrenic human subject, wherein the method which comprises orally administering a therapeutically effective amount of the composition to the subject. In an embodiment, the composition is formulated for oral delivery and the therapeutically effective amount is a total daily dose of Compound I of between about 1 mg to about 64 mg. In an embodiment, the therapeutically effective amount is a total daily dose of Compound I of between about 10 mg to about 64 mg, 20 mg to about 64 mg, or about 30 mg to about 64 mg. In an embodiment, the therapeutically effective amount is a total daily dose of Compound I of about 8 mg, about 16 mg, about 32 mg or about 64 mg.

In another aspect, the disclosure provides a method of treating at least one negative symptom in a non-schizophrenic human subject, wherein the method comprises administering to the subject a therapeutically effective amount of Compound I, or a pharmaceutically acceptable salt, hydrate, solvate or polymorph thereof. In an embodiment, the method comprises orally administering a total daily dose of Compound I of between about 1 mg and about 64 mg. In an embodiment, the total daily dose of Compound I is between about 10 mg to about 64 mg, 20 mg to about 64 mg, or about 30 mg to about 64 mg. In an embodiment, the total daily dose of Compound I is about 8 mg, about 16 mg, about 32 mg or about 64 mg.

In both aspects of the disclosure, the negative symptom to be treated is a primary negative symptom rather than a secondary negative symptom. In an embodiment, the primary negative symptom is selected from the group consisting of: blunted affect, alogia, amotivation, anhedonia and asociality. In an embodiment, the primary negative symptom is selected from the group consisting of: blunted affect, emotional withdrawal, poor rapport, passive/apathetic social withdrawal, difficulty in abstract thinking, lack of spontaneity and flow of conversation, and stereotyped thinking.

In some embodiments of any of the above aspects of the disclosure, the non-schizophrenic patient is diagnosed with a mental disorder or a neurological condition. In an embodiment, the mental disorder or neurological condition is selected from the group consisting of: dementia, frontotemporal dementia (FTD), Alzheimer's disease, autism spectrum disorder (ASD), bipolar disorder (BPD), major depressive disorder (MDD), borderline personality disorder, Parkinson's disease, temporal lobe epilepsy, post-cerebrovascular accident (CVA), traumatic brain injury (TBI), post brain trauma syndrome, mild to moderate mental retardation, viral encephalitis, and drug addiction. In an embodiment, the disorder or condition is FTD or Alzheimer's disease. In an embodiment, the disorder or condition is MDD or BPD. In an embodiment, the disorder or condition is Parkinson's disease.

In some embodiments of any of the above aspects of the disclosure, Compound I or a pharmaceutically acceptable salt, hydrate, solvate or polymorph thereof is administered to the subject for a first treatment period of sufficient length to achieve improvement in at least one negative symptom. In an embodiment, the first treatment period is at least 2 weeks, at least 4 weeks, at least 6 weeks, at least 8 weeks, at least 10 weeks or at least 12 weeks.

In some embodiments of any of the above aspects of the disclosure, if a subject experiences improvement in at least one negative symptom during the first treatment period, then administration of the therapeutically effective dose of Compound I or a pharmaceutically acceptable salt, hydrate, solvate or polymorph thereof is continued for a second treatment period of at least 12 weeks, at least 24 weeks, at least 48 weeks or until the subject is determined to be in remission from the negative symptoms.

In some embodiments of any of the above aspects of the disclosure, Compound I or a pharmaceutically acceptable salt, hydrate, solvate or polymorph thereof is administered in a single dose in the morning or evening. In an embodiment, Compound I or a pharmaceutically acceptable salt, hydrate, solvate or polymorph thereof is administered at least two hours before eating.

In some embodiments of any of the above aspects of the disclosure, a polymorph of Compound I is administered to the subject. In an embodiment, the polymorph is known as Form (A) of Compound (I)·HCl·2H₂O (also referred to herein as Form (A)) and has the characteristics described in the international patent application PCT/US2015/062985 (published as WO 2016/089766) and US patent application U.S. Ser. No. 14/954,264 (published as US 2016-0152597 A1), each of which was filed on 30 Nov. 2015, the contents of which are incorporated by reference in their entirety.

In some embodiments of any of the above aspects of the disclosure, Compound I or polymorph Form (A) is administered as part of a pharmaceutical composition which comprises a release modifier that provides a maximum plasma concentration (Cₘₐₓ) of Compound (I) or polymorph Form (A) below 50 ng/mL when a dose of about 1 mg to about 64 mg of the formulation is administered to a human. In an embodiment, the pharmaceutical composition provides a maximum plasma concentration (Cₘₐₓ) for the BFB-520 metabolite of below 10.0 ng/mL, below 5.0 ng/mL, below 4.5 ng/mL, below 4.0 ng/mL, below 3.5 ng/mL, below 3.0 ng/mL, below 2.5 ng/mL, below 2.0 ng/mL, below 1.5 ng/mL, or below 1.0 ng/mL and an area under the curve (AUC) of BFB-520 below 40 hr*ng/mL, below 35 hr*ng/mL, below 30 hr*ng/mL, below 25 hr*ng/mL, below 20 hr*ng/mL, below 15 hr*ng/mL, or below 10 hr*ng/mL.

In some embodiments of any of the above aspects of the disclosure, the human subject is at least 18 years of age, while in other embodiments of any of the above aspects of the disclosure, the human subject is under 18 years of age.

In some embodiments of any of the above aspects of the disclosure, the human subject has not been previously treated with an anti-psychotic drug. In other embodiments of any of the above aspects of the disclosure, the human subject has discontinued prior treatment with an anti-psychotic drug due to experiencing an inadequate response and/or to intolerable side effects

### BRIEF DESCRIPTION OF THE DRAWINGS

The foregoing summary, as well as the following detailed description of the disclosure, will be better understood when read in conjunction with the appended drawings.

FIG. 1 is a graph illustrating the mean change from baseline in the negative subscale score of the Positive and Negative Syndrome Scale (PANSS) of the pentagonal model (Y axis) over 12 weeks of treatment with daily doses of placebo (solid), 32 mg MIN-101 (long dashes) or 64 mg MIN-101 (short dashes).

FIG. 2 is a graph illustrating the mean change from baseline on the PANSS three factors negative symptoms subscale over 12 weeks of treatment with daily doses of placebo (solid), 32 mg MIN-101 (long dashes) or 64 mg MIN-101 (short dashes).

FIG. 3 is a graph illustrating the mean change from baseline in the BNSS total score (Y axis) over 12 weeks of treatment with daily doses of placebo (solid), 32 mg MIN-101 (long dashes) or 64 mg MIN-101 (short dashes).

FIG. 4 is an X-ray powder diffraction of Form (A) of Compound (I)·HCl·2H₂O.

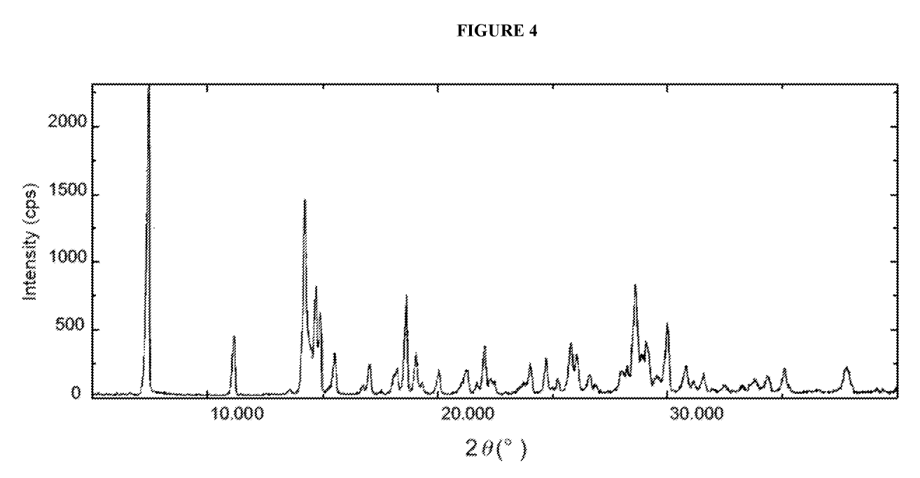

FIG. 5 is an IR spectrum of Form (A) of Compound (*)·HCl·2H₂O.

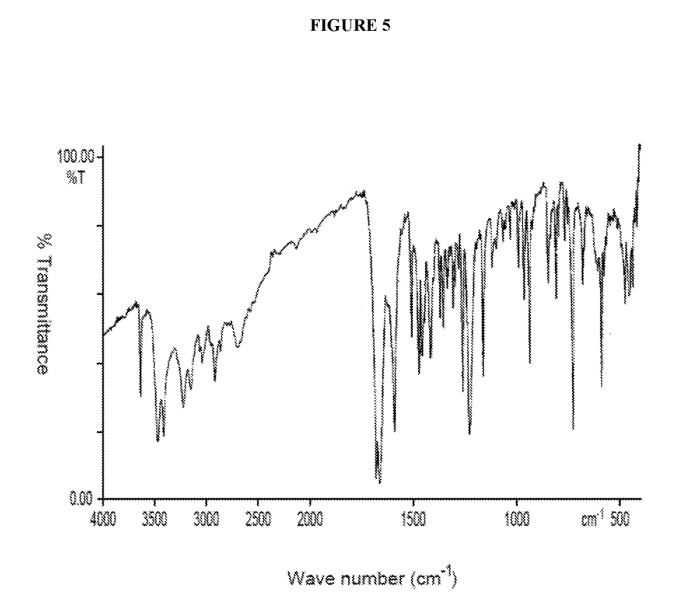

FIG. 6 is a ¹H-NMR spectrum of Form (A) of Compound (I)·HCl·2H₂O.

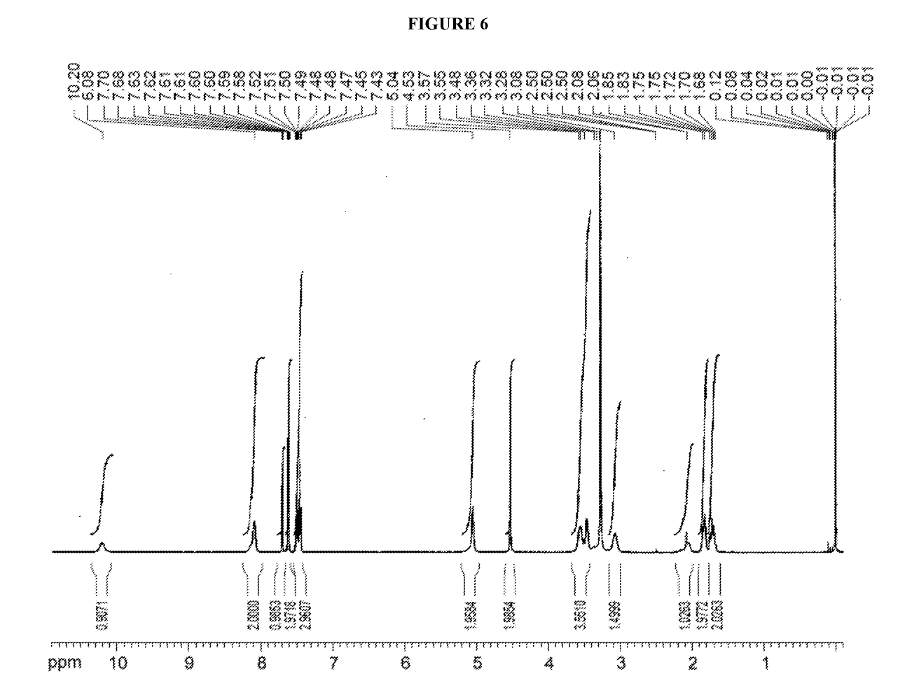

FIG. 7 is a ¹³C-NMR spectrum of Form (A) of Compound (I)·HCl·2H₂O.

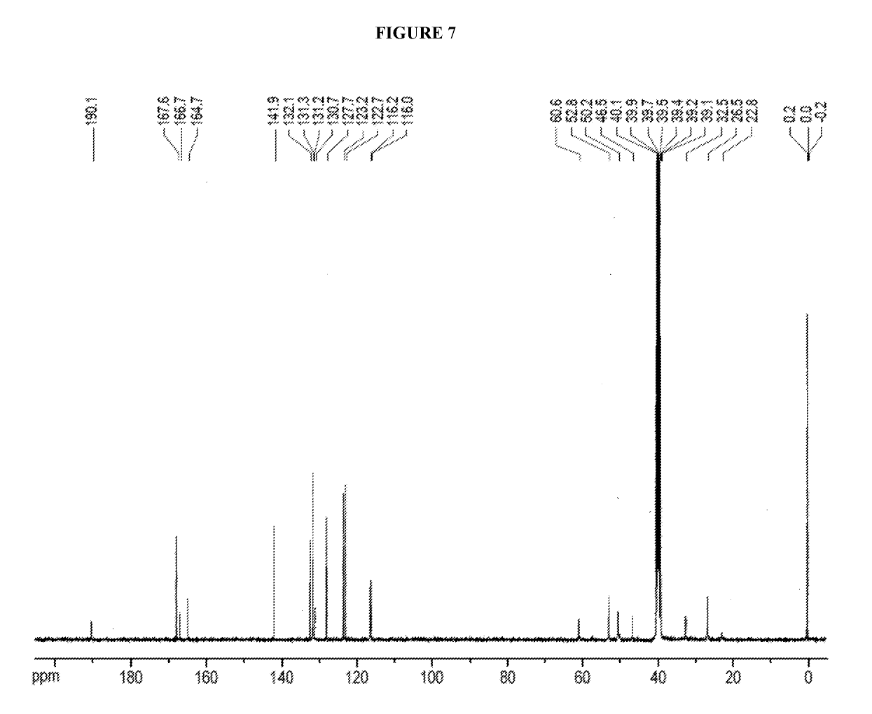

### DETAILED DESCRIPTION

As described in the Examples set forth below, 32 mg and 64 mg daily doses of Compound I have been shown to produce statistically significant improvement in negative symptoms in schizophrenic patients as compared to placebo. Based on these data, and the fact that Compound I antagonizes sigma₂ activity, the present disclosure contemplates that similar improvement in negative symptoms will be achieved in non-schizophrenic human subjects. As used herein, a non-schizophrenic subject means the subject exhibits at least one negative symptom but has not been diagnosed with schizophrenia.

Thus, it is an object of the present disclosure to provide a method of treating at least one negative symptom in a human non-schizophrenic subject comprising administering to the subject a therapeutically effective amount of a composition comprising Compound I or a pharmaceutically acceptable salt, hydrate, solvate or polymorph thereof.

It is also an object of the present disclosure to provide a composition comprising Compound I or a pharmaceutically acceptable salt, hydrate, solvate or polymorph thereof for use in treating at least one negative symptom in a human subject by a method comprising administering to the subject a therapeutically effective amount of the composition.

It is a further object of the present disclosure to employ the compositions and methods of the disclosure to treat at least one negative symptom in a human non-schizophrenic subject who is diagnosed with a mental disorder or neurological condition.

In an embodiment, the negative symptom is one of the five major sub-domains of negative symptoms: blunted affect, alogia, amotivation, anhedonia and asociality. The core characteristics of each sub-domain are described below.

Blunted affect (affective flattening, blunted expression) is characterized by reduced intensity and range of emotional expression as manifested via vocal and non-verbal modes of communication including intonation (prosody), facial expression, hand-gestures and body movements.

Alogia (poverty of speech) is characterized by decreased quantity of speech, reduced spontaneous speech and loss of conversational fluency.

Amotivation (loss of volition) is characterized by deficits in the initiation and maintenance of goal-directed behaviors like work, study, sport, personal hygiene and daily tasks, especially when requiring and effort (cognitive or physical) and significant organization, as well as deficits in desire to undertake such activities. This sub-domain is related to apathy and lack of energy.

Anhedonia (reduced ability to experience or anticipate pleasure) is characterized by the looking forward to a reward, recreational or other pleasurable experience (“wanting”) being more markedly and consistently impaired (anticipatory anhedonia) than the appreciation (“liking”) of the experience itself (consummatory anhedonia).

Asociality (social withdrawal) is characterized by diminished interest in, motivation for, and appreciation of social interactions with others, like family and friends, loss of interest in intimate (sexual) relationships independent of any somatic problems, and for a child, may include loss of interest in playing with other children.

As used herein, unless otherwise noted, the terms “treat”, “treating”, “treatment” and the like, shall include the management and care of a non-schizophrenic subject for the purpose of improving negative symptoms and include administration of Compound I in an amount and for a treatment period that are sufficient to prevent the onset of one or more negative symptoms, reduce the frequency, intensity or severity of one or more negative symptoms, delay or avoid the development of additional negative symptoms, or any combination of these treatment objectives. In an embodiment, the effect of treatment with Compound I is assessed by comparing the severity of the subject's negative symptoms at baseline (e.g., prior to treatment with Compound I) and after at least one treatment period. In an embodiment, the treatment period is at least one week, at least two weeks, at least four weeks, at least six weeks, at least eight weeks, at least 10 weeks or at least twelve weeks.

As used herein, the terms “subject” and “patient” may be used interchangeably, and refer to a human of any age. In an embodiment, the non-schizophrenic subject is six or more years of age. In some embodiments, the subject is at least 18, 19, 20 or 21 years of age. The non-schizophrenic subject exhibits one or more negative symptoms but does not have a diagnosis of schizophrenia. In some embodiments, the non-schizophrenic subject is not diagnosed with a mental disorder or neurological condition. In other embodiments, the non-schizophrenic subject is diagnosed with a mental disorder or neurological condition.

In some embodiments, a composition or method of the disclosure is used to treat a non-schizophrenic subject who is treatment-naïve to an anti-psychotic drug. As used herein, an anti-psychotic drug is any drug that does not contain Compound I and has been approved by a regulatory agency for the treatment of psychosis. Examples of atypical antipsychotics include, but are not limited to fluphenazine, risperidone, olanzapine, clozapine, quetiapine, ziprasidone, aripiprazole, sertindole, zotepine, and perospirone.

In other embodiments, a composition or method of the disclosure is used to treat a non-schizophrenic subject who was previously treated with an antipsychotic drug but discontinued such treatment, e.g., because the drug did not provide adequate improvement in the subject's negative symptoms and/or because the subject could not tolerate the side effects of the drug.

In some embodiments, a composition or method of the disclosure is used to treat a non-schizophrenic subject who is treatment-naïve to an antidepressant drug. As used herein, an antidepressant drug is any drug that does not contain Compound I and has been approved by a regulatory agency for the treatment of major depressive disorder. Examples of antidepressants include, but are not limited to, fluoxetine, citalopram, escitalopram, venlafaxine, duloxetine, and bupropion.

In other embodiments, a composition or method of the disclosure is used to treat a non-schizophrenic subject who was previously treated with an antidepressant drug but discontinued such treatment, e.g., because the drug did not provide adequate improvement in the subject's negative symptoms and/or because the subject could not tolerate the side effects of the drug.

For purposes of the disclosure encompassed herein, the term “negative symptom” or “negative symptoms” is to be understood as including primary negative symptom(s) typically associated with schizophrenia, the negative symptom(s) measured in the PANSS negative subscale score and the negative symptom(s) measured in the BNSS.

The methods of the disclosure employ administering to the subject a therapeutically effective amount of Compound I, or a pharmaceutically acceptable salt, hydrate, solvate or polymorph thereof. As used herein, the term “therapeutically effective amount” means an amount that is effective to reduce the severity of at least one negative symptom by at least 20%, at least 30%, at least 40%, at least 50%, or at least 60% compared to baseline. Improvement in symptoms in the subject may be measured using any measurement tool generally accepted in the art, including but not limited to the PANSS negative subscale score of the pentagonal model or the Brief Negative Symptom Scale (BNSS) as described herein. In an embodiment, the therapeutically effective amount results in a reduction in the PANSS negative subscale from baseline of >20% after 2 weeks, 4 weeks, or 8 weeks of treatment.

In yet another aspect of the disclosure, a composition of the disclosure is formulated and administered to the subject in a manner that provides a dose of Compound I that is substantially equivalent to oral administration of any of the total daily doses specifically described herein. The skilled artisan can readily select formulations and administration routes that would provide such functional equivalence.

The disclosure also provides use of Compound I or a pharmaceutically acceptable salt, hydrate, solvate or polymorph thereof, in the manufacture of a medicament for treating at least one negative symptom in a non-schizophrenic human subject. For example, the medicament is suitable for oral administration. For example, the medicament has a therapeutically effective amount of Compound I, which corresponds to a total daily dose of Compound I of between about 1 mg to about 64 mg.

It will be understood by the skilled artisan that the treating physician may select a dose and dosing regimen within the above guidelines that he or she believes is appropriate based on the health and condition of the subject to be treated, as well as the desired outcome of the treatment. For example, the treating physician may choose to start therapy with a lower than therapeutically effective dose of Compound I and titrate up to a target therapeutically effective dose. For example, the total daily dose of Compound I may be administered in a single dose or in multiple doses.

As used herein, quantitative expressions recited as a range of from about value X to about value Y include any value that is 10% higher or lower than each of X and Y, and also includes any numerical value that falls between X and Y. Thus, for example, a dose of about 32 mg includes a dose of between 30 to 34 mg.

All references to Compound I herein include all pharmaceutically acceptable salts and all solvates and alternative physical forms thereof unless otherwise stated. All doses recited herein are based on the weight of the free base of Compound I, rather than the pharmaceutically acceptable salt, hydrate of solvate thereof or any excipients in the composition, unless otherwise stated. Further, all doses of Compound I recited herein are flat doses (e.g., not dependent on weight of the patient) unless otherwise stated.

For therapeutic administration according to the present disclosure, Compound I may be employed in the form of its free base, but is preferably used in the form of a pharmaceutically acceptable salt. In an embodiment, the form of Compound I used in the compositions and methods of the disclosure is 2-{1-[2-(4-Fluorophenyl)-2-oxoethyl]piperidin-4-ylmethyl}-2,3-dihydroisoindol-1-one monohydrochloride dihydrate, which has a molecular formula of C₂₂H₂₃FN₂O₂, HCl, 2H₂O and a molecular weight of 438.92.

Compound (I) may be synthesized using standard synthetic methods and procedures for the preparation of organic molecules and functional group transformations and manipulations, including the use of protective groups, as can be obtained from the relevant scientific literature or from standard reference textbooks in the field. Although not limited to any one or several sources, recognized reference textbooks of organic synthesis include: Smith, M. B.; March, J. March's Advanced Organic Chemistry: Reactions, Mechanisms, and Structure, 5^{th} ed.; John Wiley & Sons: New York, 2001; and Greene, T. W.; Wuts, P. G. M. Protective Groups in Organic Synthesis, 3^{rd}; John Wiley & Sons: New York, 1999. A method for preparing Compound (I) is described in U.S. Pat. No. 7,166,617.

The compositions and methods of the disclosure may employ Form (A) of Compound I. Pharmaceutical compositions comprising Form (A) of Compound I may be prepared as described in international patent application PCT/US2015/062985 (published as WO 2016/089766).

In an embodiment, alternative salts of Compound I with pharmaceutically acceptable acids may also be utilized in therapeutic administration, for example salts derived from the functional free base and acids including, but not limited to, palmitic acid, hydrobromic acid, phosphoric acid, acetic acid, fumaric acid, maleic acid, salicylic acid, citric acid, oxalic acid, lactic acid, malic acid, methanesulfonic acid and p-toluene sulfonic acid.

All solvates and all alternative physical forms of Compound I or its pharmaceutically acceptable derivatives as described herein, including but not limited to alternative crystalline forms, amorphous forms and polymorphs, are also within the scope of this disclosure, and all references to a compound of formula I herein (or Compound I) include all pharmaceutically acceptable salts, and all solvates and alternative physical forms thereof.

For therapeutic administration, Compound I or a pharmaceutically acceptable salt thereof, for example, the HCl salt, may be administered in pure form, but will preferably be formulated into any suitable pharmaceutically acceptable and effective composition which provides effective levels of the active ingredient in the body.

The term “pharmaceutically acceptable”, as used herein with respect to a compound or composition, refers to a form of the compound or composition that can increase or enhance the solubility or availability of the compound in a subject, in order to promote or enhance the bioavailability of the compound or composition. In an embodiment, the disclosure herein also encompasses pharmaceutically acceptable, hydrates, solvates, stereoisomers, or amorphous solids of the compounds and compositions embodied herein. For example, the term “pharmaceutically acceptable salt” is to describe a salt form of one or more of the compositions herein which are presented to increase the solubility of the compound, for example, in the gastric juices of the patient's gastrointestinal tract in order to promote dissolution and the bioavailability of the compounds and/or compositions. In an embodiment, pharmaceutically acceptable salts include those derived from pharmaceutically acceptable inorganic or organic bases and acids. Suitable salts include those derived from alkali metals such as potassium and sodium, alkaline earth metals such as calcium, magnesium and ammonium salts, among numerous other acids well known in the pharmaceutical art. Sodium and potassium salts are particularly preferred as neutralization salts of carboxylic acids and free acid phosphate containing compositions encompassed by the present disclosure. The term “salt” shall mean any salt consistent with the use of the compounds encompassed by the present disclosure. In the case where the compounds are used in pharmaceutical indications, including the treatment of depression, the term “salt” shall mean a pharmaceutically acceptable salt, consistent with the use of the compounds as pharmaceutical agents.

The term “pharmaceutically acceptable derivative” or “derivative”, as used herein, describes any pharmaceutically acceptable prodrug form (such as an ester or ether or other prodrug group) which, upon administration to a patient, provides directly or indirectly the present compound or an active metabolite of the present compound.

As set forth above, the compositions include pharmaceutically acceptable salts of the compounds in the composition. In other embodiments, the acids which are used to prepare the pharmaceutically acceptable acid addition salts of the aforementioned compounds are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)] salts, among others.

In an embodiment, compositions comprise base addition salts of the present compounds. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine (meglumine), and the lower alkanolammonium and other base salts of pharmaceutically acceptable organic amines, among others.

As used herein, the term pharmaceutically acceptable salts or complexes refers to salts or complexes (e.g., solvates, polymorphs) that retain the desired biological activity of the parent compound and exhibit minimal, if any, undesired toxicological effects. Non-limiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acids, naphthalenedisulfonic acids, and polygalacturonic acid; (b) base addition salts formed with polyvalent metal cations such as zinc, calcium, bismuth, barium, magnesium, aluminum, copper, cobalt, nickel, cadmium, sodium, potassium, and the like, or with an organic cation formed from N,N-dibenzylethylene-diamine, ammonium, or ethylenediamine; or (c) combinations of (a) and (b); e.g., a zinc tannate salt or the like.

Modifications of a compound can affect the solubility, bioavailability and rate of metabolism of the active species, thus providing control over the delivery of the active species. Further, the modifications can affect the anxiolytic activity of the compound, in some cases increasing the activity over the parent compound. This can easily be assessed by preparing the derivative and testing its activity according to the methods encompassed herein, or other methods known to those skilled in the art.

In an embodiment, the compositions may be formulated in a conventional manner using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions encompassed herein may be administered orally. In other embodiments, compositions may be administered parenterally, by inhalation spray, topically, rectally, nasally, buccally, vaginally or via an implanted reservoir. The term “parenteral” as used herein includes subcutaneous, percutaneous, intravenous, intramuscular, intra-articular, intra-synovial, intrasternal, intrathecal, intrahepatic, intralesional and intracranial injection or infusion techniques. As will be understood by the skilled artisan, in view of the embodiments encompassed herein, the dosage of active ingredient or ingredients (e.g., a compound of formula I) may be adjusted upward or downward based on the selected route of administration. Furthermore, it will be understood that optimizing the dosage of active ingredient for any selected dosage form may be desired and can be achieved by using the methods described herein or known in the art to evaluate the effectiveness of anxiolytic compounds.

The pharmaceutical compositions embodied herein may be orally administered in any orally acceptable dosage form including, but not limited to, capsules, tablets, aqueous suspensions or solutions. In the case of tablets for oral use, carriers which are commonly used include lactose and corn starch. In an embodiment, lubricating agents, such as magnesium stearate, are also added. For oral administration in a capsule form, useful diluents include lactose and/or dried corn starch, as two non-limiting examples. When aqueous suspensions are required for oral use, the active ingredient is combined with emulsifying and suspending agents. If desired, certain sweetening, flavoring or coloring agents may also be added.

The pharmaceutical compositions encompassed by the present disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In an embodiment, the therapeutically effective amount of Compound I is administered independently of any other medication that is indicated for the treatment of a mental disorder or neurological condition.

In another embodiment, the therapeutically effective amount of Compound I is administered in conjunction with one or more other medications to treat a co-morbid medical condition, including a mental disorder or neurological condition. Such other medications may be administered or co-administered in forms and dosages as known in the art, or in the alternative, as has been described above for administration of compounds of formula I. The other medication(s) may be administered before, after or simultaneously with Compound I during a desired treatment period.

### EXEMPLARY EMBODIMENTS

The present disclosure includes, but is not limited to, the following embodiments.

### Embodiment 1

A composition comprising a compound of formula I (Compound I);

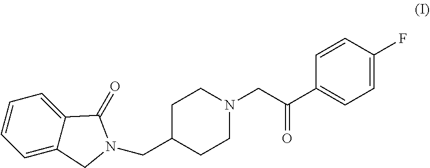 [SMILES: O=C(CN1CCC(CN2Cc3ccccc3C2=O)CC1)c1ccc(F)cc1]

or a pharmaceutically acceptable salt, hydrate, solvate or polymorph thereof, for use in a method for treating at least one negative symptom in a non-schizophrenic human subject, wherein the method comprises orally administering to the subject a therapeutically effective amount of the composition, wherein the therapeutically effective amount is a total daily dose of Compound I of between about 1 mg to about 64 mg.

### Embodiment 2

The composition of embodiment 1, wherein the therapeutically effective amount is a total daily dose of Compound I of between about 10 mg to about 64 mg, 20 mg to about 64 mg, or about 30 mg to about 64 mg.

### Embodiment 3

The composition of embodiment 2, wherein the therapeutically effective amount is a total daily dose of Compound I of between 30 mg to 64 mg.

### Embodiment 4

The composition of embodiment 1, wherein the therapeutically effective amount is a total daily dose of Compound I of about 8 mg, about 16 mg, about 32 mg or about 64 mg.

### Embodiment 5

The composition of embodiment 4, wherein the therapeutically effective amount is a total daily dose of Compound I of 32 mg.

### Embodiment 6

The composition of embodiment 4, wherein the therapeutically effective amount is a total daily dose of Compound I of 64 mg.

### Embodiment 7

A method of treating at least one negative symptom in a non-schizophrenic human subject, wherein the method comprises orally administering to the subject a therapeutically effective amount of Compound I, or a pharmaceutically acceptable salt, hydrate, solvate or polymorph thereof, wherein the therapeutically effective amount is a total daily dose of Compound I of between about 1 mg to about 64 mg.

### Embodiment 8

The method of embodiment 7, wherein the total daily dose of Compound I is between about 10 mg to about 64 mg, 20 mg to about 64 mg, or about 30 mg to about 64 mg.

### Embodiment 9

The method of embodiment 8, wherein the therapeutically effective amount is a total daily dose of Compound I of between 30 mg to 64 mg.

### Embodiment 10

The method of embodiment 7, wherein the total daily dose of Compound I is about 8 mg, about 16 mg, about 32 mg or about 64 mg.

### Embodiment 11

The method of embodiment 10, wherein the therapeutically effective amount is a total daily dose of Compound I of 32 mg.

### Embodiment 12

The method of embodiment 10, wherein the therapeutically effective amount is a total daily dose of Compound I of 64 mg.

### Embodiment 13

Use of a compound of formula I (Compound I);

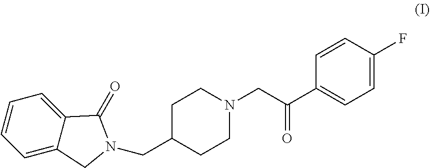 [SMILES: O=C(CN1CCC(CN2Cc3ccccc3C2=O)CC1)c1ccc(F)cc1]

or a pharmaceutically acceptable salt, hydrate, solvate or polymorph thereof, in the manufacture of a medicament for a method for treating at least one negative symptom in a non-schizophrenic human subject, wherein the method comprises orally administering to the subject a therapeutically effective amount of the composition, wherein the therapeutically effective amount is a total daily dose of Compound I of between about 1 mg to about 64 mg.

### Embodiment 14

The use of embodiment 13, wherein the therapeutically effective amount is a total daily dose of Compound I of between about 10 mg to about 64 mg, 20 mg to about 64 mg, or about 30 mg to about 64 mg.

### Embodiment 15

The use of embodiment 13, wherein the therapeutically effective amount is a total daily dose of Compound I of between 30 mg to 64 mg.

### Embodiment 16

The use of embodiment 13, wherein the therapeutically effective amount is a total daily dose of Compound I of about 8 mg, about 16 mg, about 32 mg or about 64 mg.

### Embodiment 17

The use of embodiment 16, wherein the therapeutically effective amount is a total daily dose of Compound I of 32 mg.

### Embodiment 18

The use of embodiment 16, wherein the therapeutically effective amount is a total daily dose of Compound I of 64 mg.

### Embodiment 19

The composition, method, or use of any one of embodiments 1 to 18, wherein the negative symptom is selected from the group consisting of: blunted affect, alogia, amotivation, anhedonia and asociality.

### Embodiment 20

The composition, method, or use of any one of embodiments 1 to 18, wherein the negative symptom is selected from the group consisting of: blunted affect, emotional withdrawal, poor rapport, passive/apathetic social withdrawal, difficulty in abstract thinking, lack of spontaneity and flow of conversation, and stereotyped thinking.

### Embodiment 21

The composition, method, or use of any one of embodiments 1 to 20, wherein the non-schizophrenic patient is diagnosed with a mental disorder or a neurological condition.

### Embodiment 22

The composition, method, or use of embodiment 21, wherein the mental disorder or neurological condition is selected from the group consisting of: dementia, frontotemporal dementia (FTD), Alzheimer's disease, autism spectrum disorder (ASD), bipolar disorder (BPD), major depressive disorder (MDD), Parkinson's disease, temporal lobe epilepsy, post-cerebrovascular accident (CVA), traumatic brain injury (TBI), post brain trauma syndrome, mild to moderate mental retardation, viral encephalitis, and drug addiction.

### Embodiment 23

The composition, method, or use of embodiment 22, wherein the mental disorder or neurological condition is FTD or Alzheimer's disease.

### Embodiment 24

The composition, method, or use of embodiment 22, wherein the mental disorder or neurological condition is MDD or BPD.

### Embodiment 25

The composition, method, or use of embodiment 22, wherein the mental disorder or neurological condition is Parkinson's disease.

### Embodiment 26

The composition, method, or use of any one of embodiments 1 to 25, wherein Compound I is administered to the subject for a first treatment period of at least 2 weeks, at least 4 weeks, at least 6 weeks, at least 8 weeks, at least 10 weeks or at least 12 weeks.

### Embodiment 27

The composition, method, or use of embodiment 26, wherein, if a subject experiences improvement in at least one negative symptom during the first treatment period, then administration of the therapeutically effective amount of Compound I is continued for a second treatment period of at least 12 weeks, at least 24 weeks, at least 48 weeks or until the subject is determined to be in remission from the negative symptoms.

### Embodiment 28

The composition, method, or use of any one of embodiments 1-27, wherein Compound I is administered in a single dose in the morning in fasting condition and at least two hours before eating.

### Embodiment 29

The composition, method, or use of any one of embodiments 1 to 28, wherein the polymorph Form (A) of Compound I is administered to the subject.

### Embodiment 30

The composition, method, or use of any one of embodiments 1 to 29, wherein Compound I or the polymorph Form (A) of Compound I is administered as part of a pharmaceutical composition which comprises a release modifier that provides a maximum plasma concentration (Cₘₐₓ) of Compound (I) below 50 ng/mL when a dose of about 1 mg to about 64 mg of the formulation is administered to a human.

### Embodiment 31

The composition, method, or use of any one of embodiments 29 to 30, wherein the pharmaceutical composition provides a maximum plasma concentration (Cₘₐₓ) for the BFB-520 metabolite of below 5.0 ng/mL, below 4.5 ng/mL, below 4.0 ng/mL, below 3.5 ng/mL, below 3.0 ng/mL, below 2.5 ng/mL, below 2.0 ng/mL, below 1.5 ng/mL, or below 1.0 ng/mL and an area under the curve (AUC) of BFB-520 below 40 hr*ng/mL, below 35 hr*ng/mL, below 30 hr*ng/mL, below 25 hr*ng/mL, below 20 hr*ng/mL, below 15 hr*ng/mL, or below 10 hr*ng/mL.

### Embodiment 32

The composition, method, or use of any one of embodiments 1 to 31, wherein the non-schizophrenic subject has not been previously treated with an anti-psychotic drug.

### Embodiment 33

The composition, method, or use of any one of embodiments 1 to 31, wherein the non-schizophrenic subject has discontinued prior treatment with an anti-psychotic drug due to experiencing an inadequate response and/or to intolerable side effects.

### Embodiment 34

The composition, method, or use of any one of embodiments 1 to 33, wherein the non-schizophrenic subject has not been previously treated with an anti-depressant drug.

### Embodiment 35

The composition, method, or use of any one of embodiments 1 to 33, wherein the non-schizophrenic subject has discontinued prior treatment with an anti-depressant drug due to experiencing an inadequate response and/or to intolerable side effects.

### Embodiment 36

The composition, method, or use of any one of embodiments 1 to 35, wherein the form of Compound I administered is 2-{1-[2-(4-Fluorophenyl)-2-oxoethyl]piperidin-4-ylmethyl}-2,3-dihydroisoindol-1-one monohydrochloride dihydrate.

### Embodiment 37

### The composition, method, or use of any one of embodiments 1 to 36, wherein the total daily dose of Compound I is administered in a single dose.

### Embodiment 38

### The composition, method, or use of any one of embodiments 1 to 36, wherein the total daily dose of Compound I is administered in multiple doses, e.g., twice daily or three or four times daily.

### POSITIVE AND NEGATIVE SYNDROME SCALE (PANSS)

Below is a description of the Positive and Negative Syndrome Scale (PANSS) used in the clinical study described in the Examples.

### Positive and Negative Syndrome Scale (PANSS) Rating Criteria

### General Rating Instructions

Data gathered from this assessment procedure are applied to the PANSS ratings. Each of the 30 items is accompanied by a specific definition, as well as detailed anchoring criteria for all seven rating points. These seven points represent increasing levels of psychopathology, as follows:
1—absent
2—minimal
3—mild
4—moderate
5—moderate severe
6—severe
7—extreme

In assigning ratings, one first considers whether an item is at all present, as judging by its definition. If the item is absent, it is scored 1, whereas if it is present, one must determine its severity by reference to the particular criteria from the anchoring points. The highest applicable rating point is always assigned, even if the patient meets criteria for lower points as well. In judging the level of severity, the rater must utilise a holistic perspective in deciding which anchoring point best characterises the patient's functioning and rate accordingly, whether or not all elements of the description are observed.

The rating points of 2 to 7 correspond to incremental levels of symptom severity:
A rating of 2 (minimal) denotes questionable or subtle or suspected pathology, or it also may allude to the extreme end of the normal range.
A rating of 3 (mild) is indicative of a symptom whose presence is clearly established but not pronounced and interferes little in day-to-day functioning.
A rating of 4 (moderate) characterises a symptom which, though representing a serious problem, either occurs only occasionally or intrudes on daily life only to a moderate extent.
A rating of 5 (moderate severe) indicates marked manifestations that distinctly impact on one's functioning but are not all-consuming and usually can be contained at will.
A rating of 6 (severe) represents gross pathology that is present very frequently, proves highly disruptive to one's life, and often calls for direct supervision.
A rating of 7 (extreme) refers to the most serious level of psychopathology, whereby the manifestations drastically interfere in most or all major life functions, typically necessitating close supervision and assistance in many areas.

Each item is rated in consultation with the definitions and criteria provided in this manual. The ratings are rendered on the PANSS rating form overleaf by encircling the appropriate number following each dimension.

### PANSS Rating Form

| | | | | | | Moderate | | |
|---|---|---|---|---|---|---|---|---|
| | | Absent | Minimal | Mild | Moderate | severe | Severe | Extreme |
| P1 | Delusions | 1 | 2 | 3 | 4 | 5 | 6 | 7 |
| P2 | Conceptual | 1 | 2 | 3 | 4 | 5 | 6 | 7 |
| | disorganization | | | | | | | |
| P3 | Hallucinatory behavior | 1 | 2 | 3 | 4 | 5 | 6 | 7 |
| P4 | Excitement | 1 | 2 | 3 | 4 | 5 | 6 | 7 |
| P5 | Grandiosity | 1 | 2 | 3 | 4 | 5 | 6 | 7 |
| P6 | Suspiciousness/persecution | 1 | 2 | 3 | 4 | 5 | 6 | 7 |
| P7 | Hostility | 1 | 2 | 3 | 4 | 5 | 6 | 7 |
| N1 | Blunted affect | 1 | 2 | 3 | 4 | 5 | 6 | 7 |
| N2 | Emotional withdrawal | 1 | 2 | 3 | 4 | 5 | 6 | 7 |
| N3 | Poor rapport | 1 | 2 | 3 | 4 | 5 | 6 | 7 |
| N4 | Passive/apathetic social | 1 | 2 | 3 | 4 | 5 | 6 | 7 |
| | withdrawal | | | | | | | |
| N5 | Difficulty in abstract | 1 | 2 | 3 | 4 | 5 | 6 | 7 |
| | thinking | | | | | | | |
| N6 | Lack of spontaneity & | 1 | 2 | 3 | 4 | 5 | 6 | 7 |
| | flow of conversation | | | | | | | |
| N7 | Stereotyped thinking | 1 | 2 | 3 | 4 | 5 | 6 | 7 |
| G1 | Somatic concern | 1 | 2 | 3 | 4 | 5 | 6 | 7 |
| G2 | Anxiety | 1 | 2 | 3 | 4 | 5 | 6 | 7 |
| G3 | Guilt feelings | 1 | 2 | 3 | 4 | 5 | 6 | 7 |
| G4 | Tension | 1 | 2 | 3 | 4 | 5 | 6 | 7 |
| G5 | Mannerisms & posturing | 1 | 2 | 3 | 4 | 5 | 6 | 7 |
| G6 | Depression | 1 | 2 | 3 | 4 | 5 | 6 | 7 |
| G7 | Motor retardation | 1 | 2 | 3 | 4 | 5 | 6 | 7 |
| G8 | Uncooperativeness | 1 | 2 | 3 | 4 | 5 | 6 | 7 |
| G9 | Unusual thought content | 1 | 2 | 3 | 4 | 5 | 6 | 7 |
| G10 | Disorientation | 1 | 2 | 3 | 4 | 5 | 6 | 7 |
| G11 | Poor attention | 1 | 2 | 3 | 4 | 5 | 6 | 7 |
| G12 | Lack of judgment & | 1 | 2 | 3 | 4 | 5 | 6 | 7 |
| | insight | | | | | | | |
| G13 | Disturbance of volition | 1 | 2 | 3 | 4 | 5 | 6 | 7 |
| G14 | Poor impulse control | 1 | 2 | 3 | 4 | 5 | 6 | 7 |
| G15 | Preoccupation | 1 | 2 | 3 | 4 | 5 | 6 | 7 |
| G16 | Active social avoidance | 1 | 2 | 3 | 4 | 5 | 6 | 7 |

### Scoring Instructions

Of the 30 items included in the PANSS, 7 constitute a Positive Scale, 7 a Negative Scale, and the remaining 16 a General Psychopathology Scale. The scores for these scales are arrived at by summaries of ratings across component items. Therefore, the potential ranges are 7 to 49 for the Positive and Negative Scales, and 16 to 112 for the General Psychopathology Scale. In addition to these measures, a Composite Scale is scored by subtracting the negative score from the positive score. This yields a bipolar index that ranges from −42 to +42, which is essentially a difference score reflecting the degree of predominance of one syndrome in relation to the other.

Positive Scale (P)

P1. DELUSIONS - Beliefs which are unfounded, unrealistic, and
idiosyncratic.
Basis for rating - Thought content expressed in the interview and its
influence on social relations and behavior.
1 Absent - Definition does not apply
2 Minimal - Questionable pathology; may be a the upper extreme
of normal limits
3 Mild - Presence of one or two delusions which are vague,
uncrystallized and not tenaciously held. Delusions do not
interfere with thinking, social relations or behavior.
4 Moderate - Presence of either a kaleidoscopic array of poorly
formed, unstable delusions or a few well-formed delusions that
occasionally interfere with thinking, social relations or behavior.
5 Moderate Severe - Presence of numerous well-formed delusions
that are tenaciously held and occasionally interfere with thinking,
social relations and behavior.
6 Severe - Presence of a stable set of delusions which are
crystallized, possibly systematized, tenaciously held and clearly
interfere with thinking, social relations and behavior.
7 Extreme - Presence of a stable set of delusions which are either
highly systematized or very numerous, and which dominate
major facets of the patient's life. This frequently results in
inappropriate and irresponsible action, which may even
jeopardize the safety of the patient or others.
P2. CONCEPTUAL DISORGANIZATION - Disorganized process
of thinking characterized by disruption of goal-directed sequencing,
e.g., circumstantiality, loose associations, tangentiality, gross
illogicality or thought block.
Basis for rating - Cognitive-verbal processes observed during the
course of interview.
1 Absent - Definition does not apply
2 Minimal - Questionable pathology; may be a the upper extreme
of normal limits
3 Mild - Thinking is circumstantial, tangential or paralogical. There
is some difficulty in directing thoughts towards a goal, and some
loosening of associations may be evidenced under pressure.
4 Moderate - Able to focus thoughts when communications are
brief and structured, but becomes loose or irrelevant when
dealing with more complex communications or when under
minimal pressure.
5 Moderate Severe - Generally has difficulty in organizing
thoughts, as evidenced by frequent irrelevancies,
disconnectedness or loosening of associations even when not
under pressure.
6 Severe - Thinking is seriously derailed and internally
inconsistent, resulting in gross irrelevancies and disruption of
thought processes, which occur almost constantly.
7 Extreme - Thoughts are disrupted to the point where the patient
is incoherent. There is marked loosening of associations, which
result in total failure of communication, e.g., “word salad” or
mutism.
P3. HALLUCINATORY BEHAVIOR - Verbal report or behavior
indicating perceptions which are not generated by external stimuli.
These may occur in the auditory, visual, olfactory or somatic
realms.
Basis for rating - Verbal report and physical manifestations during
the course of interview as well as reports of behavior by primary
care workers or family.
1 Absent - Definition does not apply
2 Minimal - Questionable pathology; may be a the upper extreme
of normal limits
3 Mild - One or two clearly formed but infrequent hallucinations,
or else a number of vague abnormal perceptions which do not
result in distortions of thinking or behavior.
4 Moderate - Hallucinations occur frequently but not continuously,
and the patient's thinking and behavior are only affected to a
minor extent.
5 Moderate Severe - Hallucinations occur frequently, may involve
more than one sensory modality, and tend to distort thinking
and/or disrupt behavior. Patient may have a delusional
interpretation of these experiences and respond to them
emotionally and, on occasion, verbally as well.
6 Severe - Hallucinations are present almost continuously, causing
major disruption of thinking and behavior. Patient treats these as
real perceptions, and functioning is impeded by frequent
emotional and verbal responses to them.
7 Extreme - Patient is almost totally preoccupied with
hallucinations, which virtually dominate thinking and behavior.
Hallucinations are provided a rigid interpretation and
provoke verbal and behavioral responses, including obedience to
command hallucinations.
P4. EXCITEMENT - Hyperactivity as reflected in accelerated motor
behavior, heightened responsivity to stimuli, hypervigilance or
excessive mood lability.
Basis for rating - Behavioral manifestations during the course of
interview as well as reports of behavior by primary care workers or
family.
1 Absent - Definition does not apply
2 Minimal - Questionable pathology; may be a the upper extreme
of normal limits
3 Mild - Tends to be slightly agitated, hypervigilant or mildly
overaroused throughout the interview, but without distinct
episodes of excitement or marked mood lability.
Speech may be slightly pressured.
4 Moderate - Agitation or overarousal is clearly evident throughout
the interview, affecting speech and general mobility, or episodic
outbursts occur sporadically.
5 Moderate Severe - Significant hyperactivity or frequent outbursts
of motor activity are observed, making it difficult for the patient
to sit still for longer than several minutes at any given time.
6 Severe - Marked excitement dominates the interview, delimits
attention, and to some extent affects personal functions such as
eating or sleeping.
7 Extreme - Marked excitement seriously interferes in eating and
sleeping and makes interpersonal interactions virtually
impossible. Acceleration of speech and motor activity may result
in incoherence and exhaustion.
P5. GRANDIOSITY - Exaggerated self-opinion and unrealistic
convictions of superiority, including delusions of extraordinary
abilities, wealth, knowledge, fame, power and moral righteousness.
Basis for rating - Thought content expressed in the interview and
its influence on behavior.
1 Absent - Definition does not apply
2 Minimal - Questionable pathology; may be a the upper extreme
of normal limits
3 Mild - Some expansiveness or boastfulness is evident, but
without clear-cut grandiose delusions.
4 Moderate - Feels distinctly and unrealistically superior to others.
Some poorly formed delusions about special status or abilities
may be present but are not acted upon.
5 Moderate Severe - Clear-cut delusions concerning remarkable
abilities, status or power are expressed and influence attitude but
not behavior.
6 Severe - Clear-cut delusions of remarkable superiority involving
more than one parameter (wealth, knowledge, fame, etc.) are
expressed, notably influence interactions and may be acted upon.
7 Extreme - Thinking interactions and behavior are dominated by
multiple delusions of amazing ability, wealth, knowledge, fame,
power and/or moral stature, which may take on a bizarre quality.
P6. SUSPICIOUSNESS/PERSECUTION - Unrealistic or
exaggerated ideas of persecution, as reflected in guardedness, and
distrustful attitude, suspicious hypervigilance or frank delusions
that others mean harm.
Basis for rating - Thought content expressed in the interview and
its influence on behavior.
1 Absent - Definition does not apply
2 Minimal - Questionable pathology; may be a the upper extreme
of normal limits
3 Mild - Presents a guarded or even openly distrustful attitude, but
thoughts, interactions and behavior are minimally affected.
4 Moderate - Distrustfulness is clearly evident and intrudes on the
interview and/or behavior, but there is no evidence of persecutory
delusions. Alternatively, there may be indication of loosely
formed persecutory delusions, but these do not seem to affect the
patient's attitude or interpersonal relations.
5 Moderate Severe - Patient shows marked distrustfulness, leading
to major disruption of interpersonal relations, or else there are
clear-cut persecutory delusions that have limited impact on
interpersonal relations and behavior.
6 Severe - Clear-cut pervasive delusions of persecution which may
be systematized and significantly interfere in interpersonal
relations.
7 Extreme - A network of systematized persecutory delusions
dominates the patient's thinking, social relations and behavior.
P7. HOSTILITY - Verbal and nonverbal expressions of anger and
resentment, including sarcasm, passive-aggressive behavior, verbal
abuse and assaultiveness.
Basis for rating - Interpersonal behavior observed during the course
of interview and reports by primary care workers or family.
1 Absent - Definition does not apply
2 Minimal - Questionable pathology; may be a the upper extreme
of normal limits
3 Mild - Indirect or restrained communication of anger, such as
sarcasm, disrespect, hostile expressions and occasional
irritability.
4 Moderate - Presents an overtly hostile attitude, showing frequent
irritability and direct expression of anger or resentment.
5 Moderate Severe - Patient is highly irritable and occasionally
verbally abusive or threatening.
6 Severe - Uncooperativeness and verbal abuse or threats notably
influence the interview and seriously impact upon social
relations. Patient may be violent and destructive but is not
physically assaultive towards them.
7 Extreme - Marked anger results in extreme uncooperativeness,
precluding other interactions, or in episode(s) of physical assault
towards others.

Negative Scale (N)

N1. BLUNTED AFFECT - Diminished emotional responsiveness
as characterized by a reduction in facial expression, modulation of
feelings and communicative gestures.
Basis for rating - Observation of physical manifestations of
affective tone and emotional responsiveness during the course of
interview.
1 Absent - Definition does not apply
2 Minimal - Questionable pathology; may be a the upper extreme
of normal limits
3 Mild - Changes in facial expression and communicative
gestures seem to be stilted, forced, artificial or lacking in
modulation.
4 Moderate - Reduced range of facial expression and few
expressive gestures result in a dull appearance.
5 Moderate Severe - Affect is generally ‘flat’ with only occasional
changes in facial expression and a paucity of communicative
gestures.
6 Severe - Marked flatness and deficiency of emotions exhibited
most of the time. There may be unmodulated extreme affective
discharges, such as excitement, rage or inappropriate
uncontrolled laughter.
7 Extreme - Changes in facial expression and evidence of
communicative gestures are virtually absent. Patient seems
constantly to show a barren or ‘wooden’ expression.
N2. EMOTIONAL WITHDRAWAL - Lack of interest in,
involvement with, and affective commitment to life's events.
Basis for rating - Reports of functioning from primary care
workers or family and observation of interpersonal behavior
during the course of the interview.
1 Absent - Definition does not apply
2 Minimal - Questionable pathology; may be a the upper extreme
of normal limits
3 Mild - Usually lack initiative and occasionally may show
deficient interest in surrounding events.
4 Moderate - Patient is generally distanced emotionally from the
milieu and its challenges but, with encouragement, can be
engaged.
5 Moderate Severe - Patient is clearly detached emotionally from
persons and events in the milieu, resisting all efforts of
engagement. Patient appears distant, docile, and purposeless,
but can be involved in communication at least briefly and tends
to personal needs, sometimes with assistance.
6 Severe - Marked deficiency of interest and emotional
commitment results in limited conversation with others and
frequent and frequent neglect of personal functions, for which
the patient requires supervision.
7 Extreme - Patient is almost totally withdrawn,
uncommunicative and neglectful of personal needs as a result
of profound lack of interest and emotional commitment.
N3. POOR RAPPORT - Lack of interpersonal empathy, openness
in conversation and sense of closeness, interest or involvement with
the interviewer. This is evidenced by interpersonal distancing and
reduced verbal and nonverbal communication.
Basis for rating - Interpersonal behavior during the course of
interview.
1 Absent - Definition does not apply
2 Minimal - Questionable pathology; may be a the upper extreme
of normal limits
3 Mild - Conversation is characterized by a stilted, strained or
artificial tone. It may lack emotional depth or tend to remain on
an impersonal, intellectual plane.
4 Moderate - Patient typically is aloof, with interpersonal
distance quite evident. Patient may answer questions
mechanically, act bored or express disinterest.
5 Moderate Severe - Disinvolvement is obvious and clearly
impedes the productivity of the interview. Patient may tend to
avoid eye or face contact.
6 Severe - Patient is highly indifferent, with marked interpersonal
distance. Answers are perfunctory, and there is little nonverbal
evidence of involvement. Eye and face contact are frequently
avoided.
7 Extreme - Patient is totally uninvolved with the interviewer.
Patient appears to be completely indifferent and consistently
avoids verbal and nonverbal interactions during the interview.
N4. PASSIVE/APATHETIC SOCIAL WITHDRAWAL -
Diminished interest and initiative in social interactions due to
passivity, apathy, anergy or avolition. This leads to reduced
interpersonal involvements and neglect of activities or daily living.
Basis for rating - Reports on social behavior from primary care
workers or family.
1 Absent - Definition does not apply
2 Minimal - Questionable pathology; may be a the upper extreme
of normal limits
3 Mild - Shows occasional interest in social activities but poor
initiative. Usually engages with others only when approached
first by them.
4 Moderate - Passively goes along with most social activities but
in a disinterested or mechanical way. Tends to recede into the
background.
5 Moderate Severe - Passively participates in only a minority of
activities and shows virtually no interest or initiative. Generally
spends little time with others.
6 Severe - Tends to be apathetic and isolated, participating very
rarely in social activities and occasionally neglecting personal
needs. Has very few spontaneous social contacts.
7 Extreme - Profoundly apathetic, socially isolated and
personally neglectful.
N5. DIFFICULTY IN ABSTRACT THINKING - Impairment in
the use of abstract-symbolic mode of thinking, as evidence by
difficulty in classification, forming generalizations and
proceeding beyond concrete or egocentric thinking in problem-
solving tasks.
Basis for rating - Responses to questions on similarities and
proverb interpretation, and use of concrete vs. abstract mode
during the course of the interview.
1 Absent - Definition does not apply
2 Minimal - Questionable pathology; may be a the upper extreme
of normal limits
3 Mild - Tends to give literal or personalized interpretations to
the more difficult proverbs and may have some problems with
concepts that are fairly abstract or remotely related.
4 Moderate - Often utilizes a concrete mode. Has difficulty with
most proverbs and some categories. Tends to be distracted by
functional aspects and salient features.
5 Moderate Severe - Deals primarily in a concrete mode,
exhibiting difficulty with most proverbs and many categories.
6 Severe - Unable to grasp the abstract meaning of any proverbs
or figurative expressions and can formulate classifications for
only the most simple of similarities. Thinking is either vacuous
or locked into functional aspects, salient features and
idiosyncratic interpretations.
7 Extreme - Can use only concrete modes of thinking. Shows no
comprehension of proverbs, common metaphors or similes, and
simple categories. Even salient and functional attributes do not
serve as a basis for classification. This rating may apply to
those who cannot interact even minimally with the examiner
due to marked cognitive impairment.
N6. LACK OF SPONTANEITY AND FLOW OF
CONVERSATION - Reduction in the normal flow of
communication associated with apathy, avolition, defensiveness
or cognitive deficit. This is manifested by diminished fluidity and
productivity of the verbal interactional process.
Basis for rating - Cognitive-verbal processes observed during the
course of interview.
1 Absent - Definition does not apply
2 Minimal - Questionable pathology; may be a the upper extreme
of normal limits
3 Mild - Conversation shows little initiative. Patient's answers
tend to be brief and unembellished, requiring direct and leading
questions by the interviewer.
4 Moderate - Conversation lacks free flow and appears uneven or
halting. Leading questions are frequently needed to elicit
adequate responses and proceed with conversation.
5 Moderate Severe - Patient shows a marked lack of spontaneity
and openness, replying to the interviewer's questions with only
one or two brief sentences.
6 Severe - Patient's responses are limited mainly to a few words
or short phrases intended to avoid or curtail communication.
(e.g., “I don't know”, “I'm not at liberty to say”). Conversation
is seriously impaired as a result and the interview is highly
unproductive.
7 Extreme - Verbal output is restricted to, at most, an
occasional utterance, making conversation not possible.
N7. STEREOTYPED THINKING - Decreased fluidity, spontaneity
and flexibility of thinking, as evidenced in rigid, repetitious or
barren thought content.
Basis for rating - Cognitive-verbal processes observed during the
interview.
1 Absent - Definition does not apply
2 Minimal - Questionable pathology; may be a the upper extreme
of normal limits
3 Mild - Some rigidity shown in attitude or beliefs. Patient may
refuse to consider alternative positions or have difficulty in
shifting from one idea to another.
4 Moderate - Conversation revolves around a recurrent theme,
resulting in difficulty in shifting to a new topic.
5 Moderate Severe - Thinking is rigid and repetitious to the point
that, despite the interviewer's efforts, conversation is limited to
only two or three dominating topics.
6 Severe - Uncontrolled repetition of demands, statements, ideas
or questions which severely impairs conversation.
7 Extreme - Thinking, behavior and conversation are dominated
by constant repetition of fixed ideas or limited phrases, leading
to gross rigidity, inappropriateness and restrictiveness of
patient's communication.

General Psychopathology Scale (G)

G1. SOMATIC CONCERN - Physical complaints or beliefs about
bodily illness or malfunctions.
This may range from a vague sense of ill being to clear-cut delusions
of catastrophic physical disease.
Basis for rating - Thought content expressed in the interview.
1 Absent - Definition does not apply
2 Minimal - Questionable pathology; may be a the upper extreme
of normal limits
3 Mild - Distinctly concerned about health or bodily malfunction,
but there is no delusional conviction and overconcern can be
allayed by reassurance.
4 Moderate - Complaints about poor health or bodily malfunction,
but there is no delusional conviction, and overconcern can be
allayed by reassurance.
5 Moderate Severe - Patient expresses numerous or frequent
complaints about physical illness or bodily malfunction, or else
patient reveals one or two clear-cut delusions involving these
themes but is not preoccupied by them.
6 Severe - Patient is preoccupied by one or a few clear-cut
delusions about physical disease or organic malfunction, but
affect is not fully immersed in these themes, and thoughts can
be diverted by the interviewer with some effort.
7 Extreme - Numerous and frequently reported somatic delusions,
or only a few somatic delusions of a catastrophic nature, which
totally dominate the patient's affect or thinking.
G2. ANXIETY - Subjective experience of nervousness, worry,
apprehension or restlessness, ranging from excessive concern
about the present or future to feelings of panic.
Basis for rating - Verbal report during the course of the interview
and corresponding physical manifestations.
1 Absent - Definition does not apply
2 Minimal - Questionable pathology; may be a the upper extreme
of normal limits
3 Mild - Expresses some worry, overconcern or subjective
restlessness, but no somatic and behavioral consequences are
reported or evidenced.
4 Moderate - Patient reports distinct symptoms of nervousness,
which are reflected in mild physical manifestations such as fine
hand tremor and excessive perspiration.
5 Moderate Severe - Patient reports serious problems of anxiety
which have significant physical and behavioral consequences,
such as marked tension, poor concentration, palpitations or
impaired sleep.
6 Severe - Subjective state of almost constant fear associated
with phobias, marked restlessness or numerous somatic
manifestations.
7 Extreme - Patient's life is seriously disrupted by anxiety, which
is present almost constantly and at times reaches panic proportion
or is manifested in actual panic attacks.
G3. GUILT FEELINGS - Sense of remorse or self-blame for real
or imagined misdeeds in the past.
Basis for rating - Verbal report of guilt feelings during the course
of interview and the influence on attitudes and thoughts.
1 Absent - Definition does not apply
2 Minimal - Questionable pathology; may be a the upper
extreme of normal limits
3 Mild - Questioning elicits a vague sense of guilt or self-blame
for a minor incident, but the patient clearly is not overly
concerned.
4 Moderate - Patient expresses distinct concern over his
responsibility for a real incident in his life but is not pre-
occupied with it and attitude and behavior are essentially
unaffected.
5 Moderate Severe - Patient expresses a strong sense of guilt
associated with self-depreciation or the belief that he deserves
punishment. The guilt feelings may have a delusional basis, may
be volunteered spontaneously, may be a source of preoccupation
and/or depressed mood, and cannot be allayed readily by the
interviewer.
6 Severe - Strong ideas of guilt take on a delusional quality and
lead to an attitude of hopelessness or worthlessness. The
patient believes he should receive harsh sanctions such as
punishment.
7 Extreme - Patient's life is dominated by unshakable delusions
or guilt, for which he feels deserving of drastic punishment,
such as life imprisonment, torture, or death.
There may be associated suicidal thoughts of attribution of
others' problems to one's own past misdeeds.
G4. TENSION - Overt physical manifestations of fear, anxiety,
and agitation, such as stiffness, tremor, profuse sweating and
restlessness.
Basis for rating - Thought content expressed in the interview.
1 Absent - Definition does not apply
2 Minimal - Questionable pathology; may be a the upper
extreme of normal limits
3 Mild - Posture and movements indicate slight apprehensiveness,
such as minor rigidity, occasional restlessness, shifting of
positions, or fine rapid hand tremor.
4 Moderate - A clearly nervous appearance emerges from various
manifestations, such as fidgety behavior, obvious hand tremor,
excessive perspiration, or nervous mannerisms.
5 Moderate Severe - Pronounced tension is evidenced by
numerous manifestations, such as nervous shaking, profuse
sweating and restlessness, but conduct in the interview is not
significantly affected.
6 Severe - Pronounced tension to the point that interpersonal
interactions are disrupted. The patient, for example, may be
constantly fidgeting, unable to sit still for long, or show
hyperventilation.
7 Extreme - Marked tension is manifested by signs of panic or
gross motor acceleration, such as rapid restless pacing and
inability to remain seated for longer than a minute,
which makes sustained conversation not possible.
G5. MANNERISMS AND POSTURING - Unnatural movements
or posture as characterized by an awkward, stilted, disorganized,
or bizarre appearance.
Basis for rating - Verbal report during the course of the interview
and corresponding physical manifestations.
1 Absent - Definition does not apply
2 Minimal - Questionable pathology; may be a the upper extreme
of normal limits
3 Mild - Slight awkwardness in movements or minor rigidity of
posture.
4 Moderate - Movements are notably awkward or disjointed, or
an unnatural posture is maintained for brief periods.
5 Moderate Severe - Occasional bizarre rituals or contorted
posture are observed, or an abnormal position is sustained for
extended periods.
6 Severe - Frequent repletion of bizarre rituals, mannerisms or
stereotyped movements, or a controlled posture is sustained
for extended periods.
7 Extreme - Functioning is seriously impaired by virtually
constant involvement in ritualistic, manneristic, or stereotyped
movements or by an unnatural fixed posture which is
sustained most of the time.
G6. DEPRESSION - Feelings of sadness, discouragement,
helplessness and pessimism.
Basis for rating - Verbal report of depressed mood during the
course of interview and its observed influence on attitudes and
behavior.
1 Absent - Definition does not apply
2 Minimal - Questionable pathology; may be a the upper
extreme of normal limits
3 Mild - Expresses some sadness or discouragement only on
questioning, but there is no evidence of depression in general
attitude or demeanor.
4 Moderate - Distinct feelings of sadness or hopelessness,
which may be spontaneously divulged, but depressed mood
has no major impact on behavior or social functioning and the
patient usually can be cheered up.
5 Moderate Severe - Distinctly depressed mood is associated
with obvious sadness, pessimism, loss of social interest,
psychomotor retardation and some interference in appetite and
sleep. The patient cannot be easily cheered up.
6 Severe - Markedly depressed mood is associated with
sustained feelings of misery, occasional crying, hopelessness
and worthlessness. In addition, there is major
interference in appetite and/or sleep as well as in normal
motor and social functions, with possible signs of self-neglect.
7 Extreme - Depressive feelings seriously interfere in most
major functions. The manifestations include frequent crying,
pronounced somatic symptoms, impaired concentration,
psychomotor retardation, social disinterest, self neglect,
possible depressive or nihilistic delusions and/or possible
suicidal thoughts or action.
G7. MOTOR RETARDATION - Reduction in motor activity
as reflected in slowing or lessening or movements and speech,
diminished responsiveness of stimuli, and reduced body tone.
Basis for rating - Manifestations during the course of interview
as well as reports by primary care workers as well as family.
1 Absent - Definition does not apply
2 Minimal - Questionable pathology; may be a the upper
extreme of normal limits
3 Mild - Slight but noticeable diminution in rate of movements
and speech. Patient may be somewhat underproductive in
conversation and gestures.
4 Moderate - Patient is clearly slow in movements, and speech
may be characterized by poor productivity including long
response latency, extended pauses or slow pace.
5 Moderate Severe - A marked reduction in motor activity
renders communication highly unproductive or delimits
functioning in social and occupational situations.
Patient can usually be found sitting or lying down.
6 Severe - Movements are extremely slow, resulting in a
minimum of activity and speech. Essentially the day is spent
sitting idly or lying down.
7 Extreme - Patient is almost completely immobile and virtually
unresponsive to external stimuli.
G8. UNCOOPERATIVENESS - Activity refusal to comply with
the will of significant others, including the interviewer, hospital
staff or family, which may be associated with distrust,
defensiveness, stubbornness, negativism, rejection of authority,
hostility or belligerence.
Basis for rating - Verbal report during the course of the interview
and corresponding physical manifestations.
1 Absent - Definition does not apply
2 Minimal - Questionable pathology; may be a the upper
extreme of normal limits
3 Mild - Complies with an attitude of resentment, impatience,
or sarcasm. May offensively object to sensitive probing during
the interview.
4 Moderate - Occasional outright refusal to comply with normal
social demands, such as making own bed, attending scheduled
programs, etc. The patient may project a hostile, defensive or
negative attitude but usually can be worked with.
5 Moderate Severe - Patient frequently is incompliant with the
demands of his milieu and may be characterized by others as
an “outcast” or having “a serious attitude problem”.
Uncooperativeness is reflected in obvious defensiveness or
irritability with the interviewer and possible unwillingness to
address many questions.
6 Severe - Patient is highly uncooperative, negativistic and
possibly also belligerent.
Refuses to comply with most social demands and may be
unwilling to initiate or conclude the full interview.
7 Extreme - Active resistance seriously impacts on virtually all
major areas of functioning. Patient may refuse to join in any
social activities, tend to personal hygiene, converse with
family or staff and participate even briefly in an interview.
G9. UNUSUAL THOUGHT CONTENT - Thinking characterized
by strange, fantastic or bizarre ideas, ranging from those which are
remote or atypical to those which are distorted, illogical and
patently absurd.
Basis for rating - Thought content expressed during the course of
interview.
1 Absent - Definition does not apply
2 Minimal - Questionable pathology; may be a the upper
extreme of normal limits
3 Mild - Thought content is somewhat peculiar, or idiosyncratic,
or familiar ideas are framed in an odd context.
4 Moderate - Ideas are frequently distorted and occasionally seem
quite bizarre.
5 Moderate Severe - Patient expresses many strange and fantastic
thoughts, (e.g., Being the adopted son of a king, being an
escapee from death row), or some which are patently absurd
(e.g., Having hundreds of children, receiving no radio message
from outer space from a tooth filling).
6 Severe - Patient expresses many illogical or absurd ideas or
some which have a distinctly bizarre quality (e.g., having three
heads, being a visitor from another planet.
7 Extreme - Thinking is replete with absurd, bizarre and
grotesque ideas.
G10. DISORIENTATION - Lack of awareness of one's relationship
to the milieu, including persons, place and time, which may be due
to confusion or withdrawal.
Basis for rating - Responses to interview questions on orientation.
1 Absent - Definition does not apply
2 Minimal - Questionable pathology; may be a the upper extreme
of normal limits
3 Mild - General orientation is adequate but there is some
difficulty with specifics. For example, patient knows his
location but not the street address, knows hospital staff
names but not their functions, knows the month but confuses
the day of the week with an adjacent day, or errs in the date by
more than two days. There may be narrowing of interest
evidenced by familiarity with the immediate but not extended
milieu, such as ability to identify staff but not the mayor,
governor, or president.
4 Moderate - Only partial success in recognizing persons, places,
and time. For example, patient knows he is in a hospital but not
its name, knows the name of the city but not the borough or
distinct, knows the name of his primary therapist but not many
other direct care workers, knows the year or season but not sure
of the month.
5 Moderate Severe - Considerable failure in recognizing persons,
place and time.
Patient has only a vague notion of where he is and seems
unfamiliar with most people in his milieu. He may identify the
year correctly or nearly but not know the current month, day of
week or even the season.
6 Severe - Marked failure in recognizing persons, place, and time.
For example, patient has no knowledge of his whereabouts,
confuses the date by more than one year, can name only one or
two individuals in this current life.
7 Extreme - Patient appears completely disoriented with regard to
persons, place, and time. There is gross confusion or total
ignorance about one's location, the current year and even the
most familiar people, such as parents, spouse, friends, and
primary therapist.
G11. POOR ATTENTION - Failure in focused alertness manifested
by poor concentration, distractibility for internal and external stimuli,
and difficulty in harnessing, sustaining or shifting focus to new
stimuli.
Basis for rating - Manifestations during the course of interview.
1 Absent - Definition does not apply
2 Minimal - Questionable pathology; may be a the upper extreme
of normal limits
3 Mild - Limited concentration evidenced by occasional
vulnerability to distraction and faltering attention toward the
end of the interview.
4 Moderate - Conversation is affected by the tendency to be
easily distracted, difficulty in long sustaining concentration on a
given topic, or problems in shifting attention to new topics.
5 Moderate Severe - Conversation is seriously hampered by poor
concentration, distractibility, and difficulty in shifting focus
appropriately.
6 Severe - Patient's attention can be harnessed for only brief
moments or with great effort, due to marked distraction by
internal or external stimuli.
7 Extreme - Attention is so disrupted that even brief conversation
is not possible.
G12. LACK OF JUDGMENT AND INSIGHT- Impaired awareness
or understanding of one's own psychiatric condition and life
situation. This is evidenced by failure to recognize past or present
psychiatric illness or symptoms, denial of need for psychiatric
hospitalization or treatment, decisions characterized by poor
anticipation or consequences, and unrealistic short-term and long-
range planning
Basis for rating - Thought content expressed during the interview.
1 Absent - Definition does not apply
2 Minimal - Questionable pathology; may be a the upper extreme
of normal limits
3 Mild - Recognizes having a psychiatric disorder but clearly
underestimates its seriousness, the implications for treatment, or
the importance of taking measures to avoid relapse. Future
planning may be poorly conceived.
4 Moderate - Patient shows only a vague or shallow recognition
of illness. There may be fluctuations in acknowledgement of
being ill or little awareness of major symptoms which are
present, such as delusions, disorganized thinking,
suspiciousness and social withdrawal. The patient may
rationalize the need for treatment in terms of its relieving
lesser symptoms, such as anxiety, tension and sleep difficulty.
5 Moderate Severe - Acknowledges past but not present
psychiatric disorder. If challenged, the patient may concede the
presence of some unrelated or insignificant symptoms, which
tend to be explained away by gross misinterpretation or
delusional thinking. The need for psychiatric treatment similarly
goes unrecognized
6 Severe - Patient denies ever having had a psychiatric disorder.
He disavows the presence of any psychiatric symptoms in the
past or present and, though compliant, denies the need for
treatment and hospitalization.
7 Extreme - Emphatic denial of past and present psychiatric
illness. Current hospitalization and treatment are given a
delusional interpretation (e.g., as punishment for misdeeds, as
persecution by tormentors, etc.), and the patient thus refuses to
cooperate with therapists, medication or other aspects of
treatment.
G13. DISTURBANCE OF VOLITION - Disturbance in the willful
initiation, sustenance and control of one's thoughts, behavior,
movements, and speech
Basis for rating - Thought content and behavior manifested in the
course of interview.
1 Absent - Definition does not apply
2 Minimal - Questionable pathology; may be a the upper extreme
of normal limits
3 Mild - There is evidence of some indecisiveness in conversation
and thinking, which may impede verbal and cognitive processes
to a minor extent.
4 Moderate - Patient is often ambivalent and shows clear
difficulty in reaching decisions. Conversation may be marred
by alteration in thinking, and in consequence, verbal and
cognitive functioning is clearly impaired.
5 Moderate Severe - Disturbance of volition interferes in thinking
as well as behavior.
Patient shows pronounced indecision that impedes the initiation
and continuation of social and motor activities, and which also
may be evidenced in halting speech.
6 Severe - Disturbance of volition interferes in the execution of
simple automatic motor functions, such as dressing or
grooming, and markedly affects speech.
7 Extreme - Almost complete failure of volition is manifested by
gross inhibition of movement and speech resulting in
immobility and/or mutism.
G14. POOR IMPULSE CONTROL - Disordered regulation and
control of action on inner urges, resulting in sudden, unmodulated,
arbitrary or misdirected discharge of tension and emotions without
concern about consequences.
Basis for rating - Behavior during the course of interview and
reported by primary care workers or family.
1 Absent - Definition does not apply
2 Minimal - Questionable pathology; may be a the upper extreme
of normal limits
3 Mild - Patient tends to be easily angered and frustrated when
facing stress or denied gratification but rarely acts on impulse.
4 Moderate - Patient gets angered and verbally abusive with
minimal provocation.
May be occasionally threatening, destructive, or have one or
two episodes involving physical confrontation or a minor brawl.
5 Moderate Severe - Patient exhibits repeated impulsive episodes
involving verbal abuse, destruction of property, or physical
threats. There may be one or two episodes involving serious
assault, for which the patient requires isolation, physical
restraint, or PRN sedation.
6 Severe - Patient frequently impulsive aggressive, threatening,
demanding, and destructive, without any apparent consideration
of consequences. Shows assaultive behavior and may also be
sexually offensive and possibly respond behaviorally to
hallucinatory commands
7 Extreme - Patient exhibits homicidal, sexual assaults, repeated
brutality, or self-destructive behavior. Requires constant direct
supervision or external constraints because of inability to
control dangerous impulses.
G15. PREOCCUPATION - Absorption with internally generated
thoughts and feelings and with autistic experiences to the
detriment of reality orientation and adaptive behavior.
Basis for rating - Interpersonal behavior observed during the
course of interview.
1 Absent - Definition does not apply
2 Minimal - Questionable pathology; may be a the upper extreme
of normal limits
3 Mild - Excessive involvement with personal needs or problems,
such that conversation veers back to egocentric themes and
there is diminished concern exhibited toward others.
4 Moderate - Patient occasionally appears self-absorbed, as if
daydreaming or involved with internal experiences, which
interferes with communication to a minor extent.
5 Moderate Severe - Patient often appears to be engaged in
autistic experiences, as evidence by behaviors that significantly
intrude on social and communicational functions, such as the
presence of a vacant stare, muttering or talking to oneself, or
involvement with stereotyped motor patterns.
6 Severe - Marked preoccupation with autistic experiences,
which seriously delimits concentration, ability to converse, and
orientation to the milieu. The patient frequently may be
observed smiling, laughing, muttering, talking, or shouting to
himself.
7 Extreme - Gross absorption with autistic experiences, which
profoundly affects all major realms of behavior. The patient
constantly may be responding verbally or behaviorally to
hallucinations and show little awareness of other people or the
external milieu.
G16. ACTIVE SOCIAL AVOIDANCE - Diminished social
involvement associated with unwarranted fear, hostility, or
distrust.
Basis for rating - Reports of social functioning from primary care
workers or family.
1 Absent - Definition does not apply
2 Minimal - Questionable pathology; may be a the upper extreme
of normal limits
3 Mild - Patient seems ill at ease in the presence of others and
prefers to spend time alone, although he participates in social
functions when required.
4 Moderate - Patient begrudgingly attends all or most social
activities but may need to be persuaded or may terminate
prematurely on account of anxiety, suspiciousness, or hostility.
5 Moderate Severe - Patient fearfully or angrily keeps away from
many social interactions despite others' efforts to engage him.
Tends to spend unstructured time alone.
6 Severe - Patient participates in very few social activities
because of fear, hostility, or distrust. When approached, the
patient shows a strong tendency to break off interactions, and
generally he tends to isolate himself from others.
Extreme - Patient cannot be engaged in social activities because
of pronounced fears, hostility, or persecutory delusions. To the
extent possible, he avoids all interactions and remains isolated
from others.

Brief Negative Symptom Scale

Below is a description of the Brief Negative Symptom Scale (BNSS) used in the clinical study described in the Examples.

BRIEF NEGATIVE SYMPTOM SCALE

| | ←NORMAL IMPAIRED→ | | | | | | | |
|---|---|---|---|---|---|---|---|---|
| I. ANHEDONIA | | | | | | | | |
| 1. INTENSITY OF PLEASURE | 0 | 1 | 2 | 3 | 4 | 5 | 6 | |
| DURING ACTIVITIES | | | | | | | | |
| 2. FREQUENCY OF PLEASURE | 0 | 1 | 2 | 3 | 4 | 5 | 6 | |
| DURING ACTIVITIES | | | | | | | | |
| 3. INTENSITY OF EXPECTED | 0 | 1 | 2 | 3 | 4 | 5 | 6 | |
| PLEASURE FROM FUTURE | | | | | | | | |
| ACTIVITIES | | | | | | | | |
| II. DISTRESS | | | | | | | | |
| 4. DISTRESS | 0 | 1 | 2 | 3 | 4 | 5 | 6 | |
| III. ASOCIALITY | | | | | | | | |
| 5. ASOCIALITY: BEHAVIOR | 0 | 1 | 2 | 3 | 4 | 5 | 6 | |
| 6. ASOCIALITY: INTERNAL | 0 | 1 | 2 | 3 | 4 | 5 | 6 | |
| EXPERIENCE | | | | | | | | |
| IV. AVOLITION | | | | | | | | |
| 7. AVOLITION: BEHAVIOR | 0 | 1 | 2 | 3 | 4 | 5 | 6 | 9 |
| 8. AVOLITION: INTERNAL | 0 | 1 | 2 | 3 | 4 | 5 | 6 | |
| EXPERIENCE | | | | | | | | |
| V. BLUNTED AFFECT | | | | | | | | |
| 9. FACIAL EXPRESSION | 0 | 1 | 2 | 3 | 4 | 5 | 6 | |
| 10. VOCAL EXPRESSION | 0 | 1 | 2 | 3 | 4 | 5 | 6 | |
| 11. EXPRESSIVE GESTURES | 0 | 1 | 2 | 3 | 4 | 5 | 6 | |
| VI. ALOGIA | | | | | | | | |
| 12. QUANTITY OF SPEECH | 0 | 1 | 2 | 3 | 4 | 5 | 6 | 9 |
| 13. SPONTANEOUS | 0 | 1 | 2 | 3 | 4 | 5 | 6 | 9 |
| ELABORATION | | | | | | | | |

Brief Negative Symptom Scale: Manual

This rating instrument is designed to measure the current level of severity of negative symptoms in schizophrenia and schizoaffective disorder. Negative symptoms are an absence or decrease in behaviors and subjective experiences that are normally present in a person from the same culture and general age group. Negative symptoms include anhedonia, asociality, avolition, blunted affect, and alogia. Other symptoms may also belong in this group. Negative symptoms are distinct from other features of schizophrenia and related disorders, including psychotic, disorganization, mood, and anxiety symptoms, and from cognitive deficits.

The manual, which is designed for training purposes, includes description of the items, as well as probes and anchors. The Workbook, which is used when making the ratings, includes the probes and anchors only. The score sheet is a separate document.

The scale was designed for use in treatment trials, but may have other applications, including non-research clinical evaluation and tracking of change. There is no attempt to define a negative symptom subtype or syndrome in this scale. Five subscales are included, one for each of the negative symptoms listed above. There is also one other item that is not part of one of these subscales, the Distress item.

All ratings are based on a semi-structured interview with prompts and queries. It is important to include the content of the semi-structured interview as a minimum. However, you should ask any additional questions required to rate the item.

Items are rated on a 7-point (0-6) scale, with anchor points usually ranging from the symptom being absent (0) to severe (6). Ratings should be made based on the anchors, without attempting to adjust the ratings based on any expectations of how people with psychotic disorders usually perform. The time frame for the ratings is one week; ratings based on the entire lifetime of the subject are to be avoided. It may be necessary to remind the subject frequently of this timeframe. While many of the ratings in this section of the scale require self-report, observations of the subject during the interview as well as those provided by outside observers during the rating period should also be given weight, as appropriate, when generating these ratings.

For a particular item, a subject may have normal performance in some areas but clear impairment in others. In this case, a subject's rating should not be the most abnormal or the least abnormal, but an integration of the overall performance for the item; that is, the subject should receive a score that is most representative of his or her overall performance in this area. In addition, whenever there appears to be a tie between two scores on the scale, e.g. a 3 or 4, choose the lower score.

Throughout the scale, the rater should make every effort not to “carry over” ratings from one item to another within a subscale, or from one subscale to another. For instance, reduced vocal expressivity (in the Blunted Affect subscale) should not influence the rating of reduced verbal output (Poverty of Speech).

In general, ratings for Anhedonia, Asociality, and Avolition should be made on the basis of what is reasonably available to the subject. In most cases there should be some form of pleasure, socialization, and opportunities for initiative available.

I. Anhedonia Subscale

This subscale measures two different aspects of pleasure: pleasure during an activity (with intensity and frequency rated separately), and expected or anticipated pleasure from a future activity. For all three items in this subscale, consider all potential sources of pleasure to the subject, including social activities, physical sensations, recreational activities, and work/school. The rating of intensity is based on the most intense pleasure the subject has (or expects) in that area, and is based on the subject's description.

Raters should consider the pleasure associated with social activities in the Anhedonia subscale, while initiation of and persistence in social activities should be considered in the Avolition subscale.

Probe Questions: Items 1 & 2
SOCIAL: Who did you spend time with in the last week? What did you do? How did you feel when you were with him/her? How often did you spend time with them?
PHYSICAL: In the past week, was there something else that felt good physically something you smelled, tasted, or felt? If yes: How did you feel when you did that? How often did you do that?
RECREATIONAL: What have you been doing for fun in the past week? How did you feel when you did that? How often did you enjoy doing that?
WORK AND SCHOOL: Do you enjoy work (or school)? If yes: how much do you enjoy it? How often when you're working (or studying) do you enjoy it?

Item 1: Intensity of Pleasure During Activities
0. Normal: Able to enjoy fully a variety of activities; no impairment in the intensity of pleasure
1. Questionable: Enjoys activities less intensely than many people, but may still be within the range of normal.
2. Mild: A mild decrease in the intensity of pleasure in activities that is outside the range of normal.
3. Moderate: A mild decrease in the intensity of pleasure in most activities, or a moderate decrease in some.
4. Moderately severe: At least a moderate decrease in the intensity of pleasure in most activities; may have a severe decrease in one area.
5. Severe: A severe decrease in the intensity of pleasure in most activities; some ability to experience pleasure remains; may experience only mild pleasure, even in the face of what should be an intensely enjoyable experience.
6. Extremely severe: No experience of pleasure, whatever the circumstances.

Item 2: Frequency of Pleasure During Activities
0. Normal: Able to enjoy activities often; no impairment in the frequency of pleasure
1. Questionable: Enjoys activities less often than many people, but may still be within the range of normal.
2. Mild: A mild decrease in the frequency of pleasure in activities that is outside the range of normal
3. Moderate: A mild decrease in the frequency of pleasure in most activities, or a moderate decrease in some.
4. Moderately severe: At least a moderate decrease in the frequency of pleasure in most activities; may have a severe decrease in one area.
5. Severe: A severe decrease in the frequency of pleasure in most activities; some ability to experience pleasure remains, but experiences pleasure rarely, even in the face of what should be an intensely enjoyable experience.
6. Extremely severe: No experience of pleasure during the previous week.

Item 3: Intensity of Expected Pleasure from Future Activities

Probe Questions

If the subject did enjoy some activities over the past week: You said you enjoyed (list activities above). Do you expect to do any of those again soon?

If Yes: How do you think you'll feel when you do that? Are you looking forward to it?

If No: Do you want to do that again? Is there something else you would enjoy doing? (If yes: How do you think you'll feel when you do that?)

If the subject did not enjoy any activities in the past week: Are there any activities that you are looking forward to? Is there anything else you'd look forward to doing?

Some subjects may have difficulties understanding the concept of expected pleasure that is the basis of this item. This may be due to cognitive impairment, a global lack of pleasure, or some other reason. If the subjects can't understand the concept, score as a 6, and check yes in the checkbox below this item.
0. Normal: Able to experience pleasure when thinking about future activities; no impairment in the anticipation of pleasure from future activities.
1. Questionable: Less pleasure thinking about future activities than many people, but still within the range of normal.
2. Mild: A mild decrease in pleasure when thinking about future activities that is outside the range of normal.
3. Moderate: Definitely less pleasure than is normal when thinking about future activities, but does experience some pleasure.
4. Moderately severe: May experience significant pleasure thinking about some future activities, but usually does not.
5. Severe: Rarely experiences pleasure when thinking about future activities, even when considering an activity that should be very enjoyable.
6. Extremely severe: No pleasure thinking about future activities, no matter what the future activity may be.

II. Distress

This item rates the subject's experience of unpleasant or distressing emotion of any kind: sadness, depression, anxiety, grief, anger, etc. The source of the distress is not considered; for instance, unpleasant emotions associated with psychotic symptoms are considered here.

Item 4: Distress

Probe Questions

What made you feel bad in the last week? Did anything happen that you didn't like? Did anything make you feel sad or depressed? Worried or anxious? Angry or irritated?

If nothing unpleasant happened: What has happened to you in the past that made you feel bad? How do you feel about that now?
0. Normal: Normal ability to experience distress and unpleasant emotions.
1. Questionable: Less distress in the face of upsetting events than many people, but still within the range of normal.
2. Mild: Slightly less distressed than normal in the face of upsetting events.
3. Moderate: Definitely less upset than normal in the face of upsetting events, but does experience some distress.
4. Moderately severe: May experience significant distress, but usually a serious problem is necessary to elicit it.
5. Severe: Experiences only mild distress, even in the face of a serious problem.
6. Extremely severe: No experience of distress, no matter what problem is encountered.

III. Asociality Subscale

Asociality is reduced social activity accompanied by decreased interest in forming close relationships with others. This subscale is intended to capture an apathetic asociality. The item ratings are based on both reports of internal experiences, including the degree to which the subject values and desires close, social bonds, and observable behavior, namely, the extent to which the subject actually engages in interactions with others. The intent of these items is to avoid rating a suspicious withdrawal

As

Asocial behavior can include:
a) when in a social setting, engaging only superficial or brief exchanges, remaining aloof, or receding into the background
b) having no one with whom he/she discusses personal matters
c) lack of participation in events or activities with other people

Asocial internal experiences include:
a) belief that close, intimate relationships are not important or valuable
b) lack of interest in mutual caring and sharing with other people
c) preference for non-social activities
d) lack of loneliness even though isolated
e) lack of interest in cooperating and working with others

The scores for behavior and internal experience may, however, be quite different; i.e., behavior may not be congruent with internal experience. For instance, a subject may be isolated because of poor social skills or persecutory delusions—resulting in a high (i.e., impaired) score on item 5—but may feel very lonely, think about other people a great deal, and wish for companionship, resulting in a normal score on item 6.

Ratings should be made in the areas of family relationships, intimate relationships, and friendships, and if the subject does not mention anyone from each of these, the interviewer should ask about each. Interactions with the rater should also be considered in scoring this item. If the subject does not have contact with family or other social opportunities because it is not possible to have contact (because they are deceased, or they refuse to have contact with the subject), this lack of contact should not be considered in making Asociality ratings. Some allowance for the unavoidable realities of a patient's life may be necessary. For instance, a patient who is chronically institutionalized or an inpatient may not be able to have contact with family members or friends. In such a case, the rating should be based on what is available to the subject, including other patients and staff. Even in such an environment, it is possible to make connections with others, or choose not to do so, and to feel lonely or not.

Item 5: Asociality: Behavior

Probe Questions

(Refer to the people identified in items 1-3) When you spent time with them, did you contact them or did they contact you? How often do you talk to them about private, personal things? Did you try to contact anyone else?
0. No impairment: Frequently engages with others, openly discusses personal matters with one or more persons; well within normal limits.
1. Very slight deficit: Has a close relationship in which most personal matters can be discussed, actively engages with others, and has some discussion of personal matters with others.
2. Mild deficit: Social interactions are not rare, but he/she is not as active as most people; only some personal matters are discussed; relationships are not close and intimate.
3. Moderate deficit: Has no close intimate relationships with others, relationships and engagement in events are casual, but usually does not avoid others.
4. Moderately severe deficit: Contact and engagement with others tend to be rare and superficial; tends to avoid other people. Generally does not discuss personal matters with others.
5. Marked deficit: Engagement with others is almost always superficial, spends little time with others by choice.
6. Severe deficit: Rarely interacts with others, may actively avoid others most of the time.

Item 6: Asociality: Internal Experience

Probe Questions

If involved in social activities: Some people like to be by themselves; others like to be around other people. What do you prefer?

Do you feel close to (the people discussed above)? Do you think about (people discussed above) much? Do you wish you were closer? Do you feel lonely sometimes?

If not involved in social activities: Do you wish you had more contact with people? Do you think about that much? How do you feel about being alone much of the time? Are these relationships important to you?
0. No impairment: Subject is very interested in relationships, sees relationships with other people as one of the most important parts of life; if isolated, often feels lonely and wishes he or she were not alone.
1. Very slight deficit: Subject considers relationships to be important; is interested in other people; if isolated, sometimes feels lonely and wish he or she were not alone.
2. Mild deficit: Subject considers close relationships with family member(s) as somewhat important, is moderately interested in other people; is not close and intimate with others; thinks about relationships sometimes.
3. Moderate deficit: Subject considers close relationships to be of little importance; infrequently wishes he/she were closer to others.
4. Moderately severe deficit: When it comes to close relationships with others, could “take it or leave it.” Generally has no wish to discuss personal matters with others, rarely misses close relationships or wishes for closer relationships.
5. Marked deficit: Subject considers relationships to be of hardly any importance, experiences very little interest in close relationships with others, does not feel lonely.
6. Severe deficit: Subject has no interest in relationships with others, does not miss having any close relationships.

IV. Avolition Subscale

Avolition is a reduction in the initiation of and persistence in activity. The two items rate over behavior and internal experience, as a failure to initiate and persist in activity may be due to several sources that do not reflect core negative symptoms, e.g., decreased opportunity or paranoid beliefs. A subject may have a decrease in goal-directed behavior but still receive a relatively low rating on avolition if he or she has a desire to engage in such behavior. For instance, a patient who is depressed may have difficulty initiating and sustaining goal-directed behavior, and would receive a high (impaired) score on item 7. The same subject may, however, feel guilty or ashamed about his or her lack of accomplishment, frequently think about his or her obligations, and may receive a lower (more normal) score on item 8. Ratings should be based on an assessment of work, school, hobbies/recreation/pastimes, and self-care. Social activities are rated in the Asociality subscale, not in this subscale. Self-care includes grooming, washing clothes, obtaining a place to live, maintaining a household, and getting to health-related appointments; other activities may also be part of self-care. The subject should not be penalized for a lack of opportunities. For instance, it would not be appropriate to penalize a hospitalized patient for failure to seek housing if discharge from the hospital is not approaching.

In the probes below, work should be defined broadly, to include housework, child care, care of an ailing family member, etc. Similarly, if a patient has little income, or has a physical handicap, it may be difficult to participate in recreational activities, and a lack of initiation and persistence in this area may not be considered of importance.

In assessing behavior and internal experience in this area, very strong motivation and interest in one area may lead to a relatively normal rating, if this is interest accounts for much of the subject's possible time and effort. For instance, someone caring for small children may have little time for other things, and may have normal scores on the avolition subscale items if absorbed in that task.

As in asociality, some allowance for the unavoidable realities of a patient's life may be necessary; the opportunities available to the person should be considered. For instance, a patient who is chronically institutionalized or an inpatient is not able to pursue competitive employment or an education. The rating should be based on what is available to the subject, including, for instance, the activities available in a hospital ward. Even in such an environment, it is possible to find things to do, or choose not to do so, and to feel bored or not.

Item 7: Avolition: Behavior

Probe Questions

General: Tell me how you spend your time. Do you spend much time just sitting, not doing anything in particular?

Work and School:

If currently working or going to school:

How much time did you spend working (or in school or studying) this week? Do you get to there on your own? Do you wait for others to tell you what to do, or do you start the work (or schoolwork) yourself?

(If in a treatment program, and question is appropriate): Did you participate in group activities in your treatment program? If yes: Did someone encourage you to do that, or did you do it on your own?

If not currently working or going to school:

Have you looked for work or looked into taking classes in the past week? Did someone suggest it, or did you do that on your own? What are you goals?
If Yes: What did you do?
If No: Why not? [Ask to distinguish opportunity from motivation]

Recreation/hobbies/pastimes: (Consider the information on recreational activities from items 1 - 3.) Do you spend much time watching TV? (If yes to last question: Are you interested in what you watch, or are you just passing the time?)

Self-care: How often have you showered/bathed over the past week? How often did you clean your {apartment, room, house}? Did someone need to remind you do this? Does someone else remind you to do those things? (If applicable): Have you needed to look for a place to live? What have you done about that?
0. No impairment: Subject initiates and persists in work or school, recreational/hobbies/pastimes, and self-care; well within normal limits.
1. Very slight deficit: Somewhat less consistent in initiating and persisting in activities than many people, but of questionable clinical relevance.
2. Mild deficit: A mild deficit in initiating and persisting activities; for instance, may have initiated activities appropriately in the past week, but with moderate persistence; or others may have provided the initiative for activities as often as the subject did.
3. Moderate deficit: A notable deficit in initiating and persisting in activities; may not initiate activities frequently, or not persist in activities for very long; others may frequently provide the impetus for any activities.
4. Moderately severe deficit: A significant deficit in initiating; may initiate a few activities but not persist for very long. Others usually provide the impetus for any activities.
5. Marked deficit: There is an obvious lack of initiation and persistence; may initiate occasionally with little persistence in activities. Others provide nearly all of the impetus in the subject's activities.
6. Severe deficit: Nearly total lack of initiation of activities.

Item 8: Avolition: Internal Experience

Probe Questions

Work and School:

If currently working or going to school: Is your job (or school) is important to you? Do you think about it much? Do you feel motivated about it?

If not working or going to school: Do you think about getting a job or going to school? Do you miss having a job (or going to school)?
If Yes: What did you do?
If No: Why not? [Ask to distinguish opportunity from motivation]

Recreation/Hobbies/Pastimes: What do you do in your free time? What hobbies do you have? Were you thinking about these this week?

Self-care: Did you feel motivated to take care of yourself this week? (If an explanation is needed: motivated about bathing, cleaning your home, taking care of your health, etc.)
If Yes: How so?
0. No impairment: Subject feels very motivated and interested in school or work, recreational activities, and self-care; thinks about these things often, and reports he/she cares about them. Clearly normal in this area.
1. Very slight deficit: Somewhat less interested in and motivated about these things than many people, but of questionable clinical relevance.
2. Mild deficit: Subject is usually motivated in these areas, but occasionally shows a lack of interest or motivation; thinks about these things and reports caring about them a little less than is normal.
3. Moderate deficit: Subject is somewhat motivated in these areas, but also exhibits some clear deficiencies in motivation or interest; may stay in a work situation but not be interested in making any improvements, or spends little time thinking about relationships or pastimes.
4. Moderately severe deficit: Subject is only slightly motivated in these areas; only occasionally thinks about them.
5. Marked deficit: Obvious lack of interest and motivation in these areas; thinks and cares about them very little.
6. Severe deficit: Essentially no interest in these areas; does not think or care about them.

V. Blunted Affect Subscale

Blunted affect refers to a decrease in the outward expression of emotion, and the interview prompts are designed to elicit emotion. If the subject does not respond to the prompts asking about emotional experiences, this item can be rated based on the responses to other questions during the interview.

Items can be rated based on the responses to other questions during the interview.

Facial Expression

When rating facial expression, consider facial movements across all parts of the face, including in the eyes (e.g., raised brows), mouth (smiling or grimacing), and mid-face (e.g., wrinkled nose when disgusted).

Item 9: Facial Expression
0. No impairment: Well within normal limits; animated when talking of emotional experiences, with many appropriate changes in facial expressions.
1. Very slight deficit: Very slight decrease of questionable clinical relevance in the frequency and intensity of facial expressions when recounting emotional experiences.
2. Mild deficit: Mild decrease in the frequency or intensity of facial expressions; shows at least two changes in the face during the recounting of each emotional experience.
3. Moderate deficit: Notable decrease in the frequency and intensity of facial expressions, such as showing only one change in facial expression in response to each question.
4. Moderately severe deficit: Significant lack of facial expressions when recounting emotional experiences, showing facial expressions for only one or two questions; may show only three or four changes in expression in the entire conversation.
5. Marked deficit: Obvious lack of positive and negative facial expressions in response to all questions; may show only one or two slight changes in facial expression during the entire conversation.
6. Severe deficit: Total or nearly total lack of facial expressions throughout the conversation.

Vocal Expression

One component of blunted affect is modulation of the voice, which includes variation in the speed, volume, and pitch of what is spoken. The content or amount of speech is not rated here.

Item 10: Vocal Expression
0. No impairment: Normal variation in all three dimensions: speed, volume, and pitch of speech.
1. Very slight deficit: Slight decrease in one of three dimensions.
2. Mild deficit: Mild decrease in two dimensions, or moderate decrease in one dimension.
3. Moderate deficit: Moderate decrease in one dimension.
4. Moderately severe deficit: Moderate decrease in two or more dimensions, or a severe decrease in one aspect.
5. Marked deficit: Severe decrease in at least one dimension, and moderate in at least one other.
6. Severe deficit: Severe decrease in two or more dimensions (speed, volume, and pitch of speech).
9. Not rated: Subject does not speak.

Expressive Gestures

Expressive gestures include not only gestures made with the hands, but also those made with the head (e.g., nodding), shoulders (shrugging), and the trunk (e.g. leaning forward). Dyskinetic movements should not to be rated here.

Item 11: Expressive Gestures
0. No impairment: Gestures are well within normal limits; uses many gestures of the arms, hands, shoulders, head, and/or body when recounting emotional experiences.
1. Very slight deficit: Very slight decrease in the frequency of expressive gestures; of questionable clinical relevance, a slight decrement in the use of the arms, hands, head, or body.
2. Mild deficit: Mild decrease in the frequency of expressive gestures; exhibits at least two expressive gestures during the recounting of each emotional experience.
3. Moderate deficit: Notable decrease in the frequency expressive gestures; may show a slight gesture in response to each question.
4. Moderately severe deficit: Significant lack of expressive gestures exhibiting a gesture in response to only one or two of the questions; may show only three or four gestures throughout entire conversation.
5. Marked deficit: Obvious lack of expressive gestures. The reduced number of gestures occurs for all questions; may show only one or two slight gestures throughout the entire conversation.
6. Severe deficit: Nearly total lack of expressive gestures; virtually no movement of arms, hands, head or body when recounting all emotional experiences.

VI. Alogia Subscale

There are no specific probes for the Alogia subscale; ratings are based on the responses to all questions during the interview.

Quantity of Speech

This item refers to the quantity of words spoken. Other speech abnormalities, such as disorganization, neologisms, or psychotic content are not rated here. For instance, a disorganized subject may produce a large quantity of speech and have a low (normal) score on this item.

Item 12: Quantity of Speech
0. No impairment: Normal amount of speech or subject talks excessively
1. Very slight deficit: Questionable decrease in the quantity of speech; answers are typically concise.
2. Mild deficit: Answers are usually brief
3. Moderate deficit: Many answers consist of one or two words.
4. Moderately severe deficit: At least half of the replies are one or two words.
5. Marked deficit: Most answers are one or two words.
6. Severe deficit: All or nearly all replies are one or two words.
9. Not rated: Subject does not speak.

Spontaneous Elaboration

This item rates the amount of information given beyond what is strictly necessary in order to respond to the interviewer's questions. Whether or not the subject's responses are appropriate is not considered, so elaboration in this sense can include appropriate background information given to clarify an answer, irrelevant or unnecessary material, delusional or thought-disordered responses.

Item 13: Spontaneous Elaboration
0. No impairment: Subject usually provides information beyond what is needed to respond to the question; this information may or may not be appropriate; subject may even be overly talkative or have pressure of speech.
1. Very slight deficit: Subject often gives information beyond what is needed to answer the question, although at times more information might have been appropriate.
2. Mild deficit: Subject gives additional information several times, but answers are usually restricted to the information that is required.
3. Moderate deficit: Subject occasionally gives additional information; interviewer may occasionally request more detail.
4. Moderately severe deficit: Subject seldom gives information beyond what is needed to answer the question. Interviewer may request more detail several times.
5. Marked deficit: Nearly all answers provide just the information needed, or less than is required; interviewer may frequently request more information.
6. Severe deficit: No spontaneous elaboration is given at any point during the interview.
9. Not rated: Subject does not speak.

The embodiments encompassed herein are now described with reference to the following Examples. These Examples are provided for the purpose of illustration only and the disclosure encompassed herein should in no way be construed as being limited to these Examples, but rather should be construed to encompass any and all variations which become evident as a result of the teachings provided herein.

### EXAMPLES

### Example 1

### MIN-101 Improves Negative Symptoms in Schizophrenic Patients with Negative Symptoms

A prospective Phase IIb, 12-week, randomized, double-blind, placebo-controlled parallel clinical trial was conducted to evaluate the efficacy, safety and tolerability of MIN-101 in patients with negative symptoms of schizophrenia. The study was designed to evaluate the efficacy of MIN-101 monotherapy on negative symptoms using the pentagonal structure model (PSM) of the Positive and Negative Syndrome Scale (PANSS) as the primary endpoint. A total of 244 patients were randomized in equal groups to receive daily doses of MIN-101 32 mg, MIN-101 64 mg or placebo at 32 clinical sites in Russia and five European countries.

To participate in the trial, patients were required to have stable positive and negative symptoms for three months prior to entry, a PANSS negative sub-score greater than or equal to 20, and scores <4 on the following PANSS items: excitement, hyperactivity, hostility, suspiciousness, uncooperativeness and poor impulse control. The full inclusion and exclusion criteria set forth in the protocol are listed below.

Inclusion Criteria

Each potential patient must satisfy all of the following criteria before study drug administration to be enrolled in the study:
1. Patient or patient's legal representative has provided informed consent.
2. Male or female patient, 18 to 60 years of age, inclusive.
3. Patient meets the diagnostic criteria for schizophrenia as defined in the Diagnostic and Statistical Manual of Mental Disorders-Fifth Edition (DSM-5), as established by a full psychiatric interview in conjunction with the Mini International Neuropsychiatric Interview (MINI).
4. Patient is stable in terms of positive symptoms of schizophrenia over the last 3 months according to his or her treating psychiatrist.
5. Patient presents with negative symptoms of schizophrenia over the last 3 months according to his or her treating psychiatrist.
6. Patient with PANSS negative subscore of at least 20.
7. Patient with PANSS item score of <4 on:
   P4 Excitement, hyperactivity
   P7 Hostility
   P6 Suspiciousness
   G8 Uncooperativeness
   G14 Poor impulse control
8. Patients can be on any psychotropic as long as the psychotropic can be discontinued at the beginning of the washout phase without endangering the patient's safety.
9. No change in psychotropic medication during the last month (changes are allowed if done for administrative reasons or with the permission of the Sponsor's Responsible Medical Officer).
10. No history of violence against self, others, or property.
11. Patient in whom, in the opinion of the investigator, a switch to another antipsychotic medication or initiation of an antipsychotic medication is indicated.
12. Female patient, if of childbearing potential, must test negative for pregnancy and must be using a double barrier contraceptive method.
13. Patient must be extensive metabolizers for P450 CYP2D6, as determined by genotyping test before the first drug dose is administered.
14. Patient must be able to understand the nature of the study.
15. The patient is considered by the investigator to be reliable and likely to cooperate with the assessment procedures.

Exclusion Criteria

Any potential patient who meets any of the following criteria before study drug administration will be excluded from participating in the study:
1. Current bipolar disorder, panic disorder, obsessive compulsive disorder, or evidence of mental retardation.
2. Patient's condition is due to direct physiological effects of a substance (e.g., a drug of abuse, or medication) or a general medical condition.
3. Significant risk of suicide or attempted suicide, or of danger to self or others.
4. Patient has a history of substance abuse within 3 months of the Screening visit (excluding caffeine and cigarette smoking).
5. Positive urine drug screen except when related to prescribed benzodiazepines and opiates recently prescribed for an episode of acute pain (e.g., dental extraction).
6. Patient who cannot be discontinued from psychotropics other than those allowed.
7. Patient who received clozapine within 6 months of the Screening visit. [Country-specific exception: For patients in Russia, a dose of <100 mg/day for the treatment of insomnia is allowed.]
8. Patient receiving treatment with depot antipsychotic medication can be enrolled in the study 4 weeks after the last injection.
9. Patient with a history of significant other major or unstable neurological, neurosurgical (e.g., head trauma), metabolic, hepatic, renal, hematological, pulmonary, cardiovascular, metabolic, gastrointestinal, or urological disorder.
10. Patient with a history of epilepsy seizure disorder (patient with a history of childhood febrile seizure may be enrolled in this study).
11. Patient who has had electroconvulsive therapy (ECT), vagal nerve stimulation (VNS), or repetitive trans-cranial magnetic stimulation (r-TMS) within the 3 months prior to the Screening visit or who are scheduled for ECT, VNS, or r-TMS at any time during the study.
12. Patient with clinically significant abnormalities in hematology, blood chemistry, ECG, or physical examination not resolved by the Baseline visit.
13. Body Mass Index (BMI)>35.
14. Current systemic infection (e.g., Hepatitis B virus [HBV], Hepatitis C virus [HCV], human immunodeficiency virus [HIV], tuberculosis [TB]). Patients with positive Hepatitis B core antibody test and negative Hepatitis B Surface Antigen (HBsAg) may be included in the study if aminotransferase levels (alanine aminotransferase/serum glutamic pyruvic transaminase (ALT/SGPT) and aspartate aminotransferase/serum glutamic oxaloacetic transaminase (AST/SGOT) do not exceed 2 times upper limit of normal (ULN).
15. Patient who requires or may require concomitant treatment with any other medication likely to increase QT interval (e.g., paroxetine, fluoxetine, duloxetine, amiodarone).
16. Patient who requires medication inhibiting the CYP2D6.
17. Patient with a clinically significant electrocardiogram (ECG) abnormality that could be a safety issue in the study, including QT interval value corrected for heart rate using the Fridericia's formula (QTcF)>430 msec for males and >450 msec for females.
18. Patient with a history of myocardial infarction based on medical history or ECG findings at Screening.
19. Familial or personal history of long QT syndrome or with additional risk factors for torsade de Pointes (e.g., hypokalemia, hypomagnesemia).
20. Woman of child-bearing potential, or man, who are unwilling or unable to use accepted methods of birth control.
21. Woman with a positive pregnancy test, is lactating, or is planning to become pregnant during the study.
22. Patient who participated in another clinical study within 3 months prior to Screening.

All three cohorts were balanced with respect to demographic and baseline disease characteristics as shown in the Table immediately below.

| Demographic/Baseline | | | MIN-101 | | | | | | | |
|---|---|---|---|---|---|---|---|---|---|---|
| Characteristics | Placebo | | 32 mg | | 64 mg | | Total | | Overall | |
| Statistic/Category | (N = 83) | | (N = 78) | | (N = 83) | | (N = 161) | | (N = 244) | |
| Age (years) | | | | | | | | | | |
| n | 83 | | 78 | | 83 | | 161 | | 244 | |
| Mean | 40.0 | | 39.8 | | 40.6 | | 40.2 | | 40.2 | |
| SD (SE) | 10.2 | (1.1) | 10.2 | (1.2) | 10.6 | (1.2) | 10.4 | (0.8) | 10.3 | (0.7) |
| Median | 40.6 | | 40.8 | | 42.0 | | 41.0 | | 41.0 | |
| Min, Max | 21, 59.7 | | 18, 59.3 | | 19.5, 59.7 | | 18, 59.7 | | 18, 59.7 | |
| Sex | | | | | | | | | | |
| Male | 48 | (57.8%) | 41 | (52.6%) | 48 | (57.8%) | 89 | (55.3%) | 137 | (56.1%) |
| Female | 35 | (42.2%) | 37 | (47.4%) | 35 | (42.2%) | 72 | (44.7%) | 107 | (43.9%) |
| Race | | | | | | | | | | |
| Caucasian | 83 | (100.0%) | 78 | (100.0%) | 83 | (100.0%) | 161 | (100.0%) | 244 | (100.0%) |
| Other | 0 | (0.0%) | 0 | (0.0%) | 0 | (0.0%) | 0 | (0.0%) | 0 | (0.0%) |
| Height (cm) | | | | | | | | | | |
| n | 83 | | 78 | | 83 | | 161 | | 244 | |
| Mean | 172.5 | | 170.9 | | 171.5 | | 171.3 | | 171.7 | |
| SD (SE) | 8.6 | (0.9) | 9.7 | (1.1) | 8.2 | (0.9) | 8.9 | (0.7) | 8.8 | (0.6) |
| Median | 172.0 | | 170.0 | | 170.0 | | 170.0 | | 171.0 | |
| Min, Max | 153, 190 | | 150, 205 | | 156, 196 | | 150, 205 | | 150, 205 | |
| Weight (kg) | | | | | | | | | | |
| n | 83 | | 78 | | 83 | | 161 | | 244 | |
| Mean | 77.42 | | 74.16 | | 75.25 | | 74.72 | | 75.64 | |
| SD (SE) | 14.21 | (1.56) | 16.60 | (1.88) | 13.70 | (1.50) | 15.14 | (1.19) | 14.86 | (0.95) |
| Median | 78.00 | | 73.40 | | 75.00 | | 74.00 | | 75.20 | |
| Min, Max | 45.8, 107.5 | | 45, 145 | | 47.8, 120.5 | | 45, 145 | | 45, 145 | |
| BMI (kg/m\hat{ }2) | | | | | | | | | | |
| n | 83 | | 78 | | 83 | | 161 | | 244 | |
| Mean | 26.0389 | | 25.2967 | | 25.5814 | | 25.4435 | | 25.6460 | |
| SD (SE) | 4.4749 | (0.4912) | 4.4992 | (0.5094) | 4.3349 | (0.4758) | 4.4037 | (0.3471) | 4.4279 | (0.2835) |

| | | | | | |
|---|---|---|---|---|---|
| Median | 26.1710 | 24.9965 | 25.1590 | 25.1590 | 25.3760 |
| Min, Max | 17.856, 37.341 | 16.366, 34.938 | 17.414, 35.062 | 16.366, 35.062 | 16.366, 37.341 |

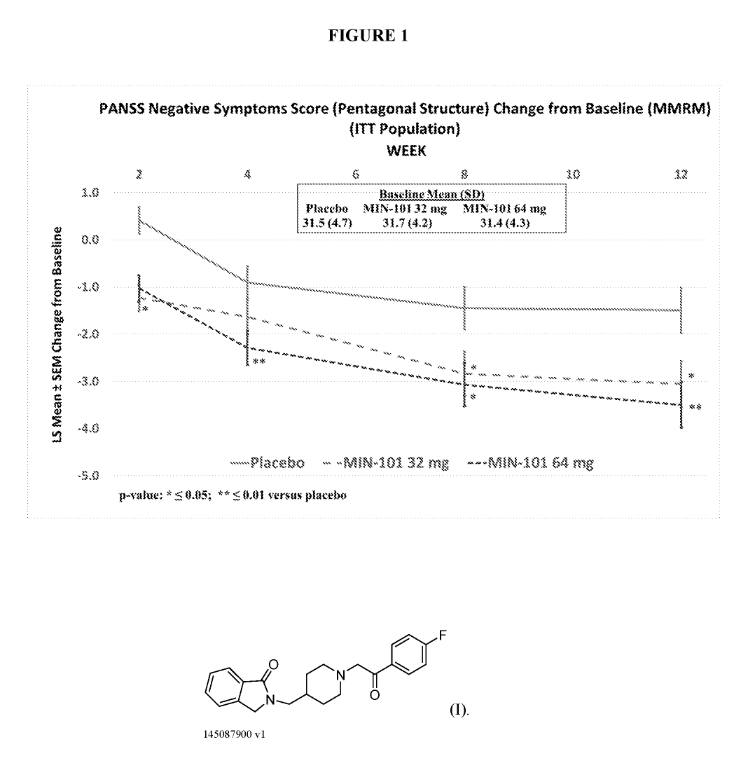

The mean changes from baseline in the PANSS Negative Subscale score in the placebo and treatment arms over 12 weeks of treatment is shown in FIG. 1. A statistically significant improvement was shown for both doses tested: 32 mg: p≤0.023 with an effect size of 0.45, and 64 mg: p≤0.003 with effect size of 0.57.

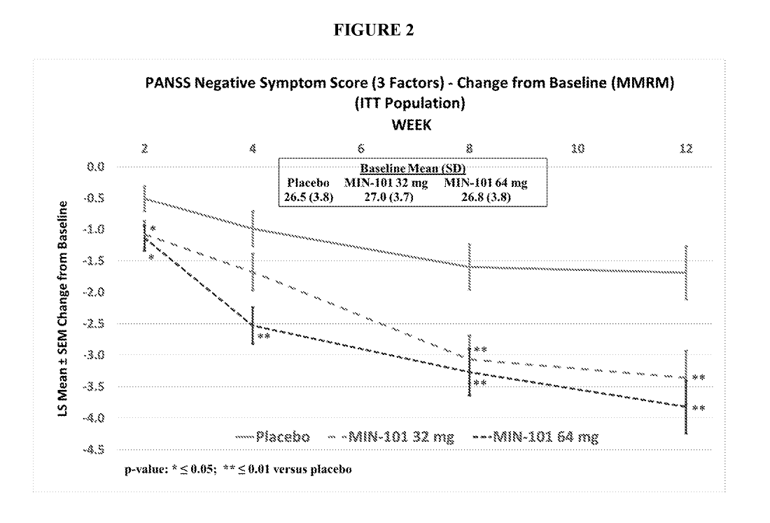

As illustrated in FIG. 2, the study also demonstrated a statistically significant benefit of MIN-101 over placebo on the PANSS three factors negative symptoms subscale for both doses tested: 32 mg: p≤0.006, with an effect size of 0.55, 64 mg: p≤0.001 with an effect size 0.70.

Furthermore, the statistically significant benefit of MIN-101 over placebo was also demonstrated on the PANSS total score (not significant for the 32 mg dose; p≤0.003 for the 64 mg dose), with effect sizes of 0.35 and 0.59, respectively.

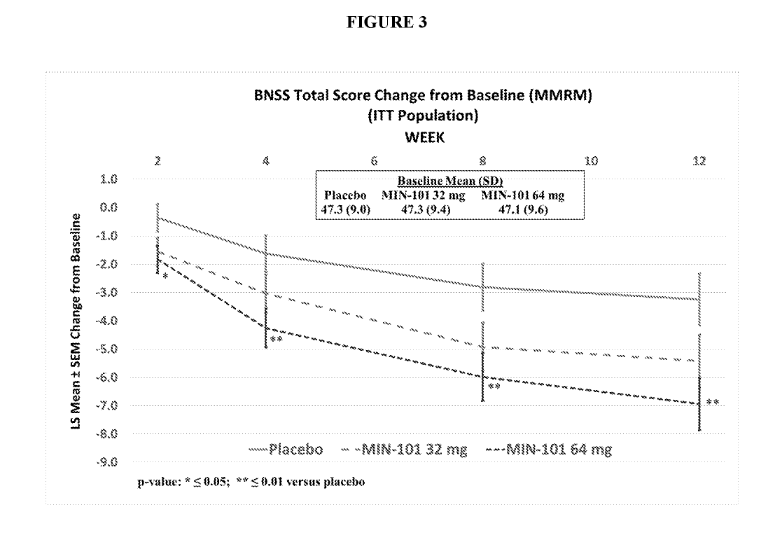

Improvement in negative symptoms achieved by both doses of MIN-101 was also observed when the effect was measured using the BNSS total score, as shown in FIG. 3.

MIN-101 was generally reported to be well tolerated, and the incidence and types of side effects did not differ significantly between the MIN-101 group and the placebo group. Based upon previous non-clinical and clinical experience, QTcF, a measurement of cardiac function, was closely monitored. Discontinuation criteria based on QTcF prolongation were incorporated in the protocol. Two patients out of 162 who received MIN-101 were discontinued based upon these criteria; both of these patients received the higher dose (64 mg). Unlike many currently marketed antipsychotic drugs, no metabolic adverse effects, no weight gain, no extra-pyramidal symptoms and no prolactin elevation were observed.

### EQUIVALENTS AND INCORPORATION BY REFERENCE

The invention has been described herein by reference to certain preferred embodiments. However, as particular variations thereon will become apparent to those skilled in the art, based on the disclosure set forth herein, the invention is not to be considered as limited thereto.

Unless otherwise defined, all technical and scientific terms used herein have the same meaning as commonly understood by one of ordinary skill in the art to which the invention belongs. In the specification and claims, the singular forms also include the plural unless the context clearly dictates otherwise.

It is to be understood that at least some of the descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Further, to the extent that the method does not rely on the particular order of steps set forth herein, the particular order of the steps should not be construed as limitation on the claims. The claims directed to the method of the present disclosure should not be limited to the performance of their steps in the order written, and one skilled in the art can readily appreciate that the steps may be varied and still remain within the spirit and scope of the present disclosure.

All patents, patent applications, references and publications cited herein are fully and completely incorporated by reference as if set forth in their entirety.

1.-10. (canceled)

11. A method of treating at least one negative symptom in a non-schizophrenic human subject, wherein the method comprises orally administering to the subject a therapeutically effective amount of Compound I: 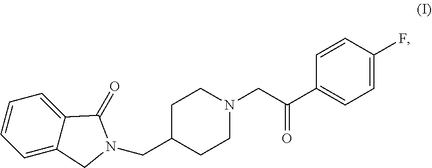 [SMILES: O=C(CN1CCC(CN2Cc3ccccc3C2=O)CC1)c1ccc(F)cc1]
or a pharmaceutically acceptable salt, hydrate, solvate or polymorph thereof, wherein the therapeutically effective amount is a total daily dose of Compound I of between about 1 mg to about 64 mg.

12. The method of claim 11, wherein the total daily dose of Compound I is between about 10 mg to about 64 mg, 20 mg to about 64 mg, or about 30 mg to about 64 mg.

13. The method of claim 11, wherein the total daily dose of Compound I is 32 mg or 64 mg.

14. The method of claim 11, wherein the negative symptom is selected from the group consisting of: blunted affect, alogia, amotivation, anhedonia and asociality.

15. The method of claim 11, wherein the negative symptom is selected from the group consisting of: blunted affect, emotional withdrawal, poor rapport, passive/apathetic social withdrawal, difficulty in abstract thinking, lack of spontaneity and flow of conversation, and stereotyped thinking.

16. The method of claim 11, wherein the non-schizophrenic patient is diagnosed with a mental disorder or a neurological condition.

17. The method of claim 16, wherein the mental disorder or neurological condition is selected from the group consisting of: dementia, frontotemporal dementia (FTD), Alzheimer's disease, autism spectrum disorder (ASD), bipolar disorder (BPD), major depressive disorder (MDD), Parkinson's disease, temporal lobe epilepsy, post-cerebrovascular accident (CVA), traumatic brain injury (TBI), post brain trauma syndrome, mild to moderate mental retardation, viral encephalitis, and drug addiction.

18. The method of claim 17, wherein the mental disorder or neurological condition is FTD, Alzheimer's disease, MDD, BPD or Parkinson's disease.

19. The method of claim 11, wherein Compound I is administered to the subject for a first treatment period of at least 2 weeks, at least 4 weeks, at least 6 weeks, at least 8 weeks, at least 10 weeks or at least 12 weeks and, if the subject experiences improvement in at least one negative symptom during the first treatment period, then administration of the therapeutically effective amount of Compound I is continued for a second treatment period of at least 12 weeks, at least 24 weeks, at least 48 weeks or until the subject is determined to be in remission from the negative symptoms.

20. The method of claim 11, wherein the non-schizophrenic subject has not been previously treated with an anti-depressant drug or has discontinued prior treatment with an anti-depressant drug due to experiencing an inadequate response and/or to intolerable side effects.
